(19) United States
(12) Patent Application Publication
(10) Pub. No.: US 2025/0058141 A1
(43) Pub. Date: Feb. 20, 2025

(54) MODERATE UV-B EXPOSURE AS A DIETARY RESTRICTION MIMETIC

(71) Applicant: Leibniz-Institut für Alternsforschung - Fritz-Lipmann-Institut e.V. (FLI), Jena (DE)

(72) Inventors: Maria Ermolaeva, Jena (DE); Asya Martirosyan, Jena (DE)

(21) Appl. No.: 18/720,600

(22) PCT Filed: Dec. 16, 2022
(86) PCT No.: PCT/EP2022/086351

§ 371 Date: Jun. 14, 2024

(30) Foreign Application Priority Data
Dec. 17, 2021 (LU) 501036

(51) Int. Cl. A61N 5/06

(52) U.S. Cl. CPC A61N 5/0613; A61N 2005/0654; A61N 2005/0661

(57) ABSTRACT

The invention relates to a method for reducing body weight of a subject comprising exposing a skin area of the subject to a sub-erythemal dose of narrow-band UV-B radiation in the range of 305-315 nm. The invention further relates to a narrow-band UV-B lamp for use in the treatment and/or prevention of obesity and/or metabolic syndrome in a subject, wherein the lamp emits UV-B radiation in the range of 305-315 nm and is preferably configured for whole body irradiation of a human subject. In another aspect the invention relates to UV-B radiation for use in the treatment and/or prevention of obesity and/or metabolic syndrome in a subject, wherein the UV-B radiation is in the range of 305-315 nm. In embodiments, the narrow-band UV-B radiation induces transient/reversible metabolic remodeling involving one or more of mitochondrial fragmentation/fission, peroxisomal and mitochondrial lipid beta-oxidation, lipid droplet turnover and glycolysis.

The invention relates to a method for reducing body weight of a subject comprising exposing a skin area of the subject to a sub-erythemal dose of narrow-band UV-B radiation, preferably in the range of 305-315 nm. The invention further relates to a narrow-band UV-B lamp for use in the treatment and/or prevention of obesity and/or metabolic syndrome in a subject, wherein the lamp emits UV-B radiation in the range of 305-315 nm and is preferably configured for whole body irradiation of a human subject. In another aspect the invention relates to UV-B radiation for use in the treatment and/or prevention of obesity and/or metabolic syndrome in a subject, wherein the UV-B radiation is in the range of 305-315 nm. In embodiments, the narrow-band UV-B radiation induces transient/reversible metabolic remodeling involving one or more of mitochondrial fragmentation/fission, peroxisomal and mitochondrial lipid beta-oxidation, lipid droplet turnover and glycolysis.

### BACKGROUND OF THE INVENTION

UV light is a common environmental factor, which affects humans regularly. UV exposure has been proposed to elicit benefits for systemic homeostasis and metabolism, but their mechanism is not well understood.

A beneficial effect of moderate UV exposure on the systemic metabolism and homeostasis has been previously discussed in the art, but its mechanism is not well understood. Most studies focus on the role of UV radiation in the synthesis of vitamin D, but this does not explain the whole spectrum of metabolic effects seen upon UV exposure (Geldenhuys et al., Diabetes, 2014 Nov;63(11):3759-69).

Ermolaeva et al. (Nature, 2013 Sep 19;501(7467):416-20) used C. elegans to uncover that transient DNA damage induced by UV-B and ionizing radiation (IR) in proliferating cells leads to systemic stress tolerance via conserved MAP kinase signaling and activation of the ubiquitin proteasome system. The contribution of mitochondria to this process has however been unexplored until today. Mitochondria are potent signaling nodes in the cell, which integrate various stress and metabolic challenges and convert them into adaptive responses via mechanisms such as metabolic remodeling, mitophagy and mitochondrial unfolded protein response (UPR^{mt}). On the one hand, mitochondria have their own genome, which can be directly impacted by treatments like UV-B and, additionally, the consumption of nicotinamide adenine dinucleotide (NAD+) by nuclear DNA damage repair machineries can influence mitochondrial function.

Previous studies identified mitochondrial fission and fusion as important mechanisms, which guide organismal adaptation to metabolic stress (Espada et. al., Nat Metab. 2020). Espada et al. show that transient impairment of mitochondrial function results in a dietary restriction-like metabolic rewiring in C. elegans, particularly it leads to a DR-like lipid turnover response, which could be measured by the whole-body Oil Red O (ORO) staining.

Ermolaeva et al., 2013 reported that nuclear DNA damage inflicted by UV-B and IR was linked to systemic stress tolerance by elevated innate immune signaling and enhanced proteostasis. Moreover, a previous study found that persistent occurrence of helix distorting nuclear DNA lesions (the damage kind induced by UV-B) leads to mitochondrial hyper-fusion and not fragmentation (Lopes at al., 2020, Nucleic Acids Res., PMID: 33021672).

US 2018/353770 A1 discloses a phototherapeutic system for treating autoimmune disorders whereby narrowband UVB (311-313 nm) is used for treating autoimmune dermatological disorders.

US 2013/203670 A1 discloses a method for treating vitiligo comprising exposing a subject to an effective amount of narrow band UVB light whereby the NB-UVB light treatment with a wavelength between 310 to 312 nm, more preferably 311 nm, as well as a repetitive exposure to UVB light is applied twice or thrice weekly.

US 2012/109042 A1 discloses a method of treating a skin condition comprising administering UV phototherapy with an excimer laser at a wavelength centered at about 308 nm.

US 2013/172963 A1 discloses a phototherapeutic apparatus which is configured to limit the vitamin D dose based on a minimum erythemal dose and has a predetermined spectrum between 290 nm and 310 nm.

WO 2015/061773 A1 discloses a method for enhancing vitamin D3 production during a phototherapy session. A UV radiation between 290 and 308 and between 280 and 320 nm is disclosed.

None of the above documents mentions application of a sub-erythemal dose of UV-B radiation for reducing body weight of a subject.

Common methods of influencing and modifying a subject's metabolism are dietary measures and physical activity. Increasing a subject's metabolic rate, for example through physical activity, might induce weight loss and reduce the levels of glucose, triglycerides and cholesterol in a subject's blood. Dietary adaptation or restriction, such as reduction of calorie intake, and physical activity are also the first line approach and standard intervention to induce weight loss for both medical and cosmetic reasons. Weight loss or body fat reduction might be desired due to cosmetic reasons if a healthy person seeks a leaner physical appearance and the reduction of excess fat tissue. Weight loss might also be chosen as a treatment of obesity and metabolic syndrome. However, these standard approaches also have several shortcomings. Dietary adaptation and physical exercise alone might not achieve in every individual the desired results in the desired time frame and/or might not be practicable for every individual aiming to lose weight.

Hence, there remains a significant need for alternative or improved approaches for inducing metabolic remodeling and weight loss in subjects that either may not desire, may not respond to or may not classify for the classical measures of dietary adaptation and exercise.

### SUMMARY OF THE INVENTION

In light of the prior art the object of the present invention is the provision of additional means for increasing the metabolic rate, the calorie and/or the body fat consumption in an individual aiming to lose weight.

This problem is solved by the features of the independent claims. Preferred embodiments of the present invention are provided by the dependent claims.

The invention therefore relates to a method for reducing body weight of a subject comprising exposing a skin area of the subject to a sub-erythemal dose of UV-B radiation.

The inventors surprisingly found that moderate UV-B exposure triggers mitochondrial fragmentation through temporary interference with mitochondrial bioenergetics function, which induces a transient metabolic restructuring response that resembles dietary restriction (DR) at both the systemic and molecular levels. The transient nature of this response is ensured by mitochondrial fusion activity, which facilitates homeostasis and stress tolerance. The unexpected effect of the present method of induction of a transient metabolic restructuring response, which resembles dietary restriction, surprisingly induces weight loss in a subject that receives moderate UV-B exposure according to the present invention. The UV-B exposure according to the invention generally aims to achieve a “sub-erythemal dose” (S.E.D.) of UV-B, which is a dose that does not cause skin (“sun”) burns in an individual subject receiving the radiation.

In preferred embodiments, the UV-B radiation is in narrow-band UV-B spectrum, preferably in a range of 305-315 nm.

Surprisingly the inventors discovered that moderate UV-B irradiation, in the wavelength range 305-315 nm, that can be emitted in embodiments from a standard “narrow-band UV-B-311” lamp, can serve in embodiments as an effective mimetic for dietary restriction to increase metabolism and hence calorie consumption and hence improve the physical appearance of a subject who desires a leaner body shape and is not satisfied with their appearance. Although ultraviolet light or phototherapy is an established form of medical and cosmetic treatment for improving the appearance of the skin in subjects suffering from conditions such as blemished skin and pimples, acne vulgaris, psoriasis and/or eczema, its prior influence on weight loss has not been known. Established prior art approach consists of irradiating a subject with narrow-band UV-B. Intriguingly, a positive effect of such cosmetic UV-B radiation on the body weight and sub-dermal fat tissue of a subject has not been known or suggested before in the prior art.

In embodiments the present method may also provide an effective mimetic for dietary restriction to increase the metabolism and calorie consumption in an individual.

It was entirely surprising that moderate narrow-band UV-B exposure can induce metabolic remodeling in a subject leading to an increased metabolic rate, which can aid the reduction of excess body fat and induce weight loss. The prior art provides no suggestions that moderate UV-B exposure might increase the metabolic rate in an individual through manipulation of mitochondrial metabolism.

The present invention is based on a further surprising finding of the role of mitochondria in the adaptive responses elicited by UV-B and IR.

Previous studies identified mitochondrial fission and fusion as important mechanisms, which guide organismal adaptation to metabolic stress (Espada et. al., Nat Metab. 2020). Espada et al. showed that transient impairment of mitochondrial function results in a dietary restriction-like metabolic rewiring in C. elegans, particularly it leads to a DR-like lipid turnover response, which could be measured by the whole-body Oil Red O (ORO) staining. In the context of the present invention, UV-B irradiation elicits transient fragmentation of mitochondria leading to the DR-like lipid reduction in whole nematodes measurable by ORO tests. A further finding is that mitochondrial fusion plays an important role in restoring homeostasis and metabolic equilibrium following UV-B treatment.

Ermolaeva et al., 2013 reported that nuclear DNA damage inflicted by UV-B and IR was linked to systemic stress tolerance by elevated innate immune signaling and enhanced proteostasis. Moreover, a previous study found that persistent occurrence of helix distorting nuclear DNA lesions (the kind of damage induced by UV-B) leads to mitochondrial hyper-fusion and not fragmentation (Lopes at al., 2020, Nucleic Acids Res., PMID: 33021672). One surprising finding of the present invention is that the signaling cascades were not influenced by metabolic UV-B effects. Altogether, these data show that metabolic and DNA damage effects of UV-B are not connected. Without being bound by theory, the present invention therefore employs biological pathways and/or mechanisms that are beneficial for metabolic remodeling, e.g., mitochondrial fragmentation, but are not connected inherently to a damaging effect on genetic elements of the subject's body.

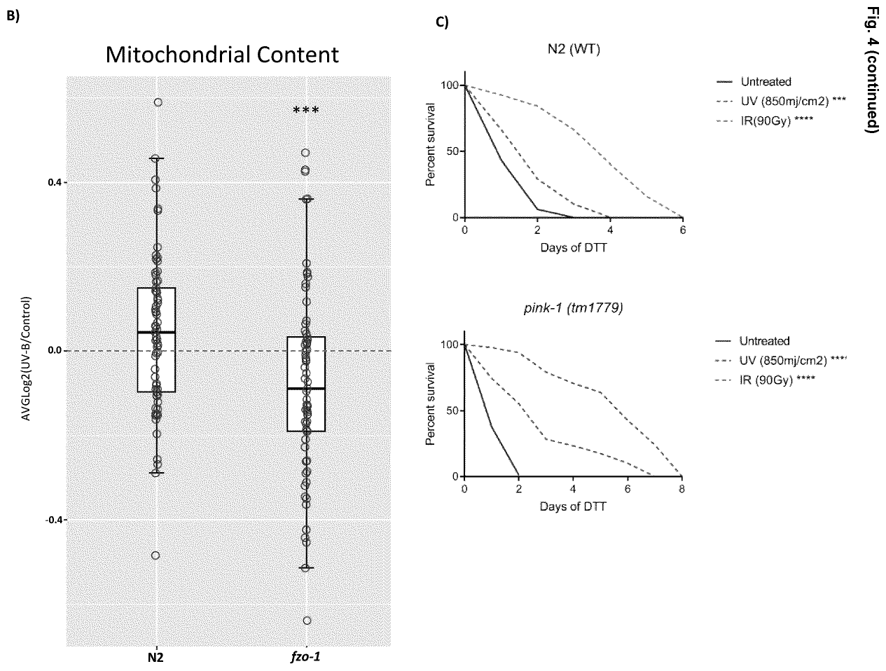

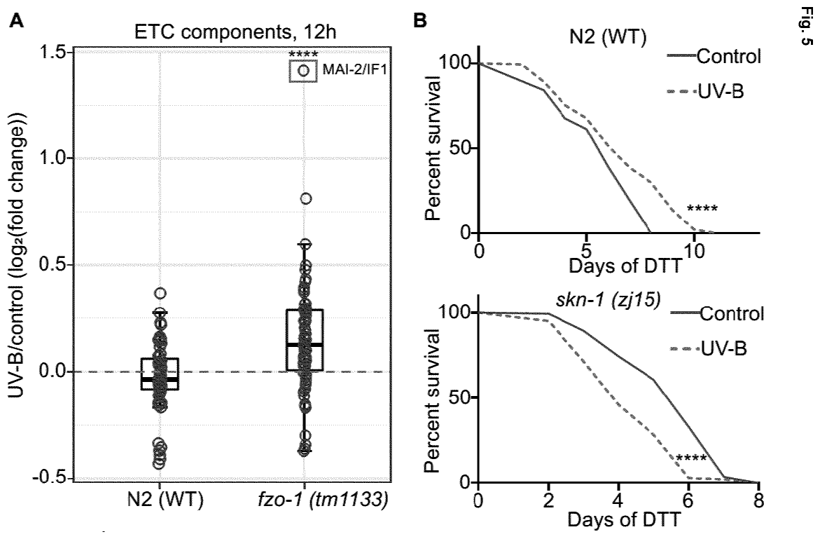

The Examples disclosed herein evidence the effect of the present invention on the model organism C. elegans, which is a standard animal model for researching the influences of radiation, e.g. UV radiation, on mitochondrial metabolism. The Examples show that UV-B with the wavelength range 305-315 nm elicits metabolic benefits by acting as a dietary restriction (DR) mimetic at the systemic and molecular levels. Mechanistically, moderate UV-B exposure with a wavelength between 305-315 nm causes a rapid disruption of the mitochondrial network. This effect is not accompanied by lasting damage of mitochondrial DNA (mtDNA) and proteins and requires fusion machinery to orchestrate the metabolic recovery process, which closely resembles DR. Importantly, the Examples demonstrate that aging-associated defects in mitochondrial fusion might abrogate systemic UV benefits in late life and might also sensitize old organisms to direct UV toxicity. Hence, in one embodiment the subject receiving UV-B irradiation according to the present invention is below 65 years old. These findings reveal that the use of UV-B irradiation according to the invention is an accessible tool, for metabolic intervention. The data in FIG. 4A-C show that UV-B irradiation in the sub-erythemal dose range according to the invention does not cause permanent cellular (mitochondrial) damage and is safe.

Hence, in certain aspects the invention also relates to a method for inducing metabolic remodelling in a subject comprising exposing a skin area of the subject to a sub-erythemal dose of narrow-band UV-B radiation in the range of 305-315 nm.

In a preferred embodiment the invention relates to a method for reducing body weight of a subject comprising exposing a skin area of the subject to a sub-erythemal dose of narrow-band UV-B radiation, wherein the subject is a human.

It was surprisingly found that in most embodiments a break of at least one day is required for the method to be effective. The inventors found that a break is in most embodiments required for restoration of metabolism after UV stress, even in healthy young subjects.

In embodiments of the invention the exposing is repeated on different days, preferably with breaks of 1-7 days, preferably until a weight loss has occurred.

In preferred embodiments, the exposing is repeated at least once, wherein between the exposing steps there is a break of at least approximately one day, such as about 12, 13, 14, 15, 16, 17, 18, 19, 20, 21, 22, 23, 24 hours. For example, it may be possible to perform one exposition in the evening of a first day, and a second in the morning of the next day. In other embodiments a longer break may be desired, wherein the length of the break between exposures may be chosen according to the skin type of the subject and/or according to the effect of the exposure event. For example, depending if one exposure event shows a strong, medium, weak or no detectable effect on the metabolism of the subject, the break between treatment may in embodiments be adjusted accordingly to influence the strength of the treatment effect and/or the time frame in which a certain effect is achieved. For example, if in one embodiment a slow reduction of body-weight or slow metabolic remodelling is desired, the breaks between treatments can be extended, if an exposure event achieves strong effects in the subject. Accordingly, if a subject shows in embodiments only a moderate or weak response to an exposure event, and a stronger and/or faster effect is desired, the breaks between the treatment may in some embodiments be shortened, as long as no physical damage is caused by the shortened UV irradiation intervals.

In embodiments of the present method the break between exposures is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95 or 100 days. In one embodiment of the present method the break between exposures is between 1-2, 1-3, 1-4, 1-5, 1-6, 1-7, 1-8, 1-9, 1-10, 1-14, 7-14, 1-21, 1-30, 1-15, 1-100 days. Preferably, 1 day is a minimal break, which is required for restoration of metabolism and/or skin recovery after UV stress in healthy young subjects. In some subjects a longer break or greater range of days between exposures may be required, especially as some “hypersensitive” subjects, e.g. subjects with a light skin type (Fitzpatrick skin type 1 or 2) or subjects that show a strong metabolic response to UV-B radiation, may require in some embodiments longer time between exposures for the restoration of metabolism and/or skin recovery. The same might apply in embodiments to older subjects. Too intensive UV exposure and/or a too short recovery time between exposures can abrogate in some embodiments the metabolic benefits of UV-B exposure, hence in embodiments the exposure dose and/or the recovery time needs to be adapted or calibrated to the individual subject, preferably to the skin type of the subject and/or age and/or metabolic responsiveness, before and/or optionally also during treatment and/or between exposures.

In embodiments of the invention the exposing is repeated on 1-7 days per week.

In embodiments of the present method the exposing is repeated on 1, 2, 3, 4, 5, 6 or 7 days per week. In embodiments the exposing is repeated on 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 days per month. In embodiments the exposing is performed every day, every other day, every 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95 or 100 days. In embodiments the exposing is performed every 1-5 days, every 2-7 days, every 3-10 days, every 7-14 days, fortnightly, every 7-27 days, every 3-7 days, every 5-10 days, every 10-20 days, every 14-30 days.

In some embodiments the maximum dose for narrow-band UV-B irradiation is 3 joules per cm² per treatment. In other embodiments the dose is 0.5, 1, 2, 4, 5, 6, 7, 8, 9, or 10 joules per cm² per treatment.

In embodiments the dose of the irradiation per treatment is correlated to the intensity of the irradiation and its duration. In some embodiments the dose of the narrow-band UV-B irradiation is regulated by the duration of exposure of the subject. In embodiments the duration per treatment can last 1-30 seconds, 1-60 seconds, 30 seconds to 5 minutes, 1-5, 1-10, 1-15, 1-20, 1-25, 1-30, 1-35, 1-40, 1-45, 1-50, 1-55, or 1-60 minutes. The duration of the irradiation is dependent on the desired dose and depends on the intensity of the radiation emitted by the irradiation source.

The low or moderate doses used in the present method are not in a range to increase the risk of skin cancer, making UV-B irradiation a safe form of cosmetic or medical treatment. Depending on the embodiment of the resent invention UV-B irradiation can be performed in a cosmetic studio, a cosmetic or medical clinic or at home. Home UV-B systems allow patients to treat themselves regularly at home.

In embodiments a success of the method of the present invention can be assumed if weight loss occurs in the subject after receival of irradiation according to the present invention. In embodiments successful metabolic remodeling can be assumed if weigh loss occurs in a subject after receival of the UV-B irradiation according to the present invention. Accordingly, weight loss is in some embodiments a suitable read-out of a successful induction of metabolic remodeling in a subject by the method according to the present invention.

In embodiments metabolic remodelling induced by the method according to the present invention in the subject can be assessed by conventional methods for determining markers in a sample taken from the subject after irradiation, preferably one or more metabolic markers, such as, blood glucose, blood cholesterol, blood LDL, blood HDL, triglyceride blood levels etc. Accordingly, the metabolic responsiveness or in other words the metabolic response of the individual subject to a UV-B exposure according to the invention may in some embodiments be assessed by determining the levels of one or more markers, such as metabolic markers in a sample from the subject.

Accordingly, in one embodiment after the exposure a sample is obtained from the individual and the level of one or more marker selected from the group consisting of serum calcium, cholesterol, HDL cholesterol, LDL cholesterol, and triglyceride levels (steady state); both steady state levels and fasting levels of serum glucose, insulin, adiponectin, leptin; Neutrophil-Lymphocyte ratio (NLR), serum interleukin (IL)-6, C-reactive protein (CRP), and interleukin (IL)-1β is determined in the sample.

In embodiments, the exposing is repeated on different days, preferably with breaks of 1-7 days, until a decrease in blood glucose, blood cholesterol, blood LDL, blood HDL, and/or blood triglycerides can be detected.

It is an important advantage of the invention that in embodiments it is not absolutely necessary to continue the treatment until a weight loss can be observed. Instead, in embodiments it is possible to detect changes of molecular markers that indicate a change in the metabolism of the subject that will lead to a reduction of body weight, also if the exposition is stopped. Such markers can be determined in a sample obtained from the subject. Different types of samples are known to the skilled person and are described in detail herein. The determination of one or more molecular markers has the advantage, that the effects of a single or several UV-B radiation exposures can be evaluated and determined immediately and further exposures can be adjusted accordingly. For example, if the frequency and/or doses and/or duration of the exposures appears to be too high, as elevated inflammation markers are detected in the sample, the breaks between the exposures and/or the intensity and/or duration of the exposure can be reduced. On the other hand, if no reduction of metabolic markers, such as blood glucose, LDL, leptin or triglycerides can be detected and/or no increase of inflammatory markers is detected the frequency and/or doses and/or duration of the exposures can be increased to achieve a stronger effect on the metabolism of the subject. Accordingly, such flexible adaptations, based on marker levels, can be done before weight loss or permanent skin damage occur and hence contribute to the safety and efficiency of embodiments of the present method.

In embodiments the effects on the metabolism of the subject receiving the sub-erythemal UV-exposure according to the present invention can be assessed by determining the levels of one or more marker selected from the group comprising metabolic markers, serum calcium, cholesterol, HDL cholesterol, LDL cholesterol, and triglyceride levels (steady state), both steady state levels and fasting levels of serum glucose, both steady state levels and fasting levels of insulin, both steady state levels and fasting levels of adiponectin and both steady state levels and fasting levels of leptin in a sample from the subject.

In some embodiments a reduction of the levels of one or more marker selected from the group comprising metabolic markers, serum calcium, cholesterol, LDL cholesterol, and triglyceride levels (steady state), serum glucose, insulin, is indicative of a desired effect of the present method on the metabolism of the subject.

In some embodiments an increase of the levels of one or more marker selected from the group comprising adiponectin, HDL cholesterol, leptin (both steady state levels and fasting levels) is indicative of a desired effect of the present method on the metabolism of the subject.

In embodiments a LDL value indicative of a healthy subject and/or a healthy metabolism may be below a value of 100-155 milligram per deciliter (mg/dl). 100 to 155 mg per deciliter (mg/dl) may be equivalent to a value below 2.6 to 4 millimole per liter (mmol/l).

In embodiments a HDL value indicative of a healthy subject and/or a healthy metabolism may be above a value of 40-50 milligram pro deciliter (mg/dl), which may be equivalent to a value above 1.03 to 1.3 millimole per liter (mmol/l).

In embodiments a triglyceride level indicative of a healthy subject and/or a healthy metabolism may be below a value of 75-150 mg/dl, preferably below 150 mg/dl, which may be equivalent to a value below 0.85 to 1.7 mmol/l.

In embodiments a serum glucose level indicative of a healthy subject and/or a healthy metabolism may be below a value of 100-140 milligram per deciliter (mg/dl), which may be equivalent to a value below 5.6 to 7.8 mmol/l.

In embodiments a serum leptin level indicative of a healthy subject and/or a healthy metabolism may be for men with a BMI of equal or below 25, between 0.3 and 10 ng/ml, while for women with the same BMI, values should be between 1 and 28 ng/ml. For men with an BMI between 26-29 (overweight, but not obese) the leptin levels should be between 1.00 and 23.0 ng/ml and for woman with the same BMI between 6.0 and 50.0 ng/ml. Leptin values considered “healthy” are usually higher in women than in men.

In embodiments a serum adiponectin indicative of a healthy subject and/or a healthy metabolism may be above a range of 7 to 10 μg/l in blood serum and/or a blood plasma level of between 10-30 μg/ml, wherein the plasma levels in women are usually higher than those of men.

In embodiments of the cosmetic method according to the invention the irradiation intensity and/or frequency of irradiation according to the invention is selected to achieve levels of one or more marker indicative of weight loss and/or metabolic remodelling in the subject, wherein the one or more markers is selected from the group comprising serum calcium, cholesterol, HDL cholesterol, LDL cholesterol, and triglyceride levels (steady state), serum glucose, insulin, adiponectin, leptin (both steady state levels and fasting levels).

In embodiments of the medical treatment method according to the invention the irradiation intensity and/or frequency of irradiation according to the invention is selected to achieve levels of one or more marker indicative of a healthy and/or normal subject, a normal metabolism, weight loss and/or metabolic remodelling in the subject, wherein the one or more markers is selected from the group comprising serum calcium, cholesterol, HDL cholesterol, LDL cholesterol, and triglyceride levels (steady state), serum glucose, insulin, adiponectin, leptin (both steady state levels and fasting levels).

In some embodiments in addition or alternatively to the afore mentioned “metabolic” markers the levels of inflammatory markers can be assessed in the subject receiving UV-B radiation according to the invention.

In embodiments, the method comprises assessing the blood or serum level of inflammatory markers, such as NLR, IL-6, CRP, and/or IL-1β. The assessment of blood NLR and serum interleukin (IL)-6, CRP, and/or IL-1β levels can serve to rule out an excessive inflammatory reaction in response to UV-B. The avoidance and/or prevention of adverse/side-effects, such as excessive inflammatory reaction and/or permanent skin damage, might be desirable in the embodiments of the cosmetic methods, as well as in the embodiments of the medical treatment methods according to the invention.

Hence, in embodiments excessive inflammatory reaction is determined or excluded by determining one or more markers selected from the group comprising blood NLR and serum interleukin (IL)-6, CRP, and/or IL-1β levels in the subject.

In some embodiments the optimal irradiation intensity and/or frequency for an Individual subject is assessed by determining of one or more markers selected from the group comprising blood NLR levels and serum levels of interleukin (IL)-6, CRP, and/or IL-1β in the subject.

In embodiments of the invention the irradiation intensity and/or frequency of irradiation according to the invention is selected to achieve levels of one or more marker indicative of the absence of excessive inflammatory reaction in the subject, wherein the one or more marker is selected from the group comprising blood NLR and serum interleukin (IL)-6, CRP, and IL-1β.

In embodiments a serum (IL)-6 level indicative of the absence of excessive inflammatory reaction may be below a value between 0.5 to 5 pg/mL.

In embodiments a serum IL-1β level indicative of of the absence of excessive inflammatory reaction may be below a value between 0.5 to 12 pg/mL.

In embodiments a serum CRP level indicative of the absence of excessive inflammatory reaction may be below a value between 1 to 10 mg/mL.

In embodiments a blood NLR indicative of the absence of excessive inflammatory reaction may be below a value between 1-3.

In embodiments the optimal irradiation intensity and/or frequency is monitored and/or assessed by determining levels of one or more markers selected from the group consisting of metabolic markers, such as serum calcium, cholesterol, HDL cholesterol, LDL cholesterol, and triglyceride levels (steady state), serum glucose, insulin, adiponectin, leptin (both steady state levels and fasting levels), and inflammatory markers, such as blood NLR and serum interleukin (IL)-6, CRP, and IL-1β.

In embodiments the one or more markers are determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95 or 100 hours or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95 and/or 100 days after UV-B exposure.

The determination of the level of one or more metabolic markers or inflammation markers can provide an understanding if the sub-erythemal UV-B exposure according to the invention is successful and can lead to the desired results. A successful exposure might be assumed, if weight loss occurs and/or if the levels of the one or more metabolic marker indicate an increased metabolic activity, an increased calorie consumption and/or the decrease of body fat. A decrease in markers, such as LDL, triglycerides, leptin and/or glucose in the blood of an individual and/or the increase of levels of HDL and/or adiponectin can indicate a desired effect of the UV-B treatment on the metabolism of the subject.

In the specific embodiments of the medical treatment method according to the invention the values of one or more markers might also move towards a range that is indicative of a normal, non-obese and healthy subject.

In some embodiments of the medical treatment method according to the invention however, it is desirable that the marker levels do not sink below thresholds for healthy individuals, which would be indicative of starvation-like excessive response. In the latter case, either the UV dose can be lowered, or the interval between treatments can be increased.

In embodiments a mild increase of inflammation markers after treatment is to be expected but the values should not get elevated above a threshold indicative of excessive inflammation response. In case the inflammatory markers are too high, in embodiments the UV dose and/or the intervals between exposures can be adjusted (dose lowered, interval increased).

The advantage of assessing the levels of one or more metabolic markers in an individual receiving the sub-erythemal UV-B exposure according to the invention is that these markers facilitate the monitoring of the effectiveness of UV-B therapy already during the time before weight loss becomes detectable. Hence, the exposure can be adjusted to either increase the efficacy of the treatment or to avoid adverse effects, such as excessive weight loss.

The advantage of assessing the levels of one or more inflammation markers in an individual receiving the sub-erythemal UV-B exposure according to the invention is that these markers facilitate the monitoring of potential adverse effects of the UV-B therapy, such as excessive inflammation or even permanent UV-induced and treatment-related physical damage in the subject.

Another aspect of the present invention relates to a sensitive biological assay which can be used to determine the appropriate dosage of irradiation for achieving this surprising effect. Hence, another aspect of the invention relates to a method for determining metabolic remodeling in a subject comprising assessing one or more markers according to the invention to adjust and personalize the treatment regimen to each individual subject.

In embodiments of the invention the exposed skin area corresponds to at least 5%, preferably at least 20%, more preferably at least 80%, of the body surface of the subject.

In embodiments of the invention the exposed skin area corresponds to at least 5%, 10%, 15%, 20%, 25%, 30%, 35%, 40%, 45%, 50%, 55%, 60%, 65%, 70%, 75%, 80%, 85%, 90%, 95%, 99% or at least 99.5% or of 100% of the body surface of the subject.

In embodiments of the invention the initial sub-erythemal dose is determined based on a skin type of the subject, wherein
a. the initial dose for a subject with
   i. Fitzpatrick skin type I is 0.2 J/cm2,
   ii. Fitzpatrick skin type II is 0.3 J/cm2,
   iii. Fitzpatrick skin type III is 0.5 J/cm2,
   iv. Fitzpatrick skin type IV to VI is 0.6 J/cm2,
b. and wherein preferably for a repeated exposure the initial dose can be increased depending on skin appearance 12-24 hours after the last exposure.

One of the aims of this specific embodiment is the establishment of the maximal (individual-specific) sub-erythemal dose for an individual subject through sequential trials. By determining the maximal sub-erythemal dose for an individual subject this embodiment has the beneficial effect of preventing adverse effects, such as excessive inflammation, skin irritation and/or skin damage, in the subject.

The exposure with too high or too frequent UV-B doses for a Fitzpatrick skin type, e.g. especially types 1 to 3, can lead to adverse reactions, such as excessive inflammation (as shown e.g. in FIG. 9 on the model organism C. elegans). Accordingly, it is important that the induction of immune response by UV-B is not exaggerated, which would be a sign of pathological cellular damage. Accordingly, it is advantageous on some embodiments to measure and monitor the immune markers in the blood of an irradiated subject, as described herein, to ensure the UV dose/treatment frequency is safe for the respective skin type of the subject.

In embodiments of the invention the subject is 65 years old or younger, preferably 50 year or younger, more preferably 40 years or younger.

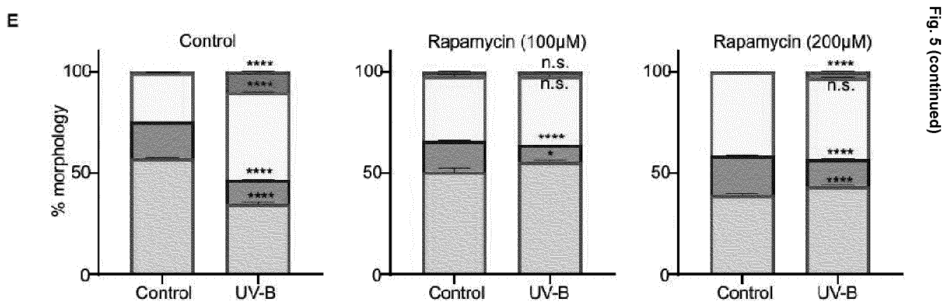

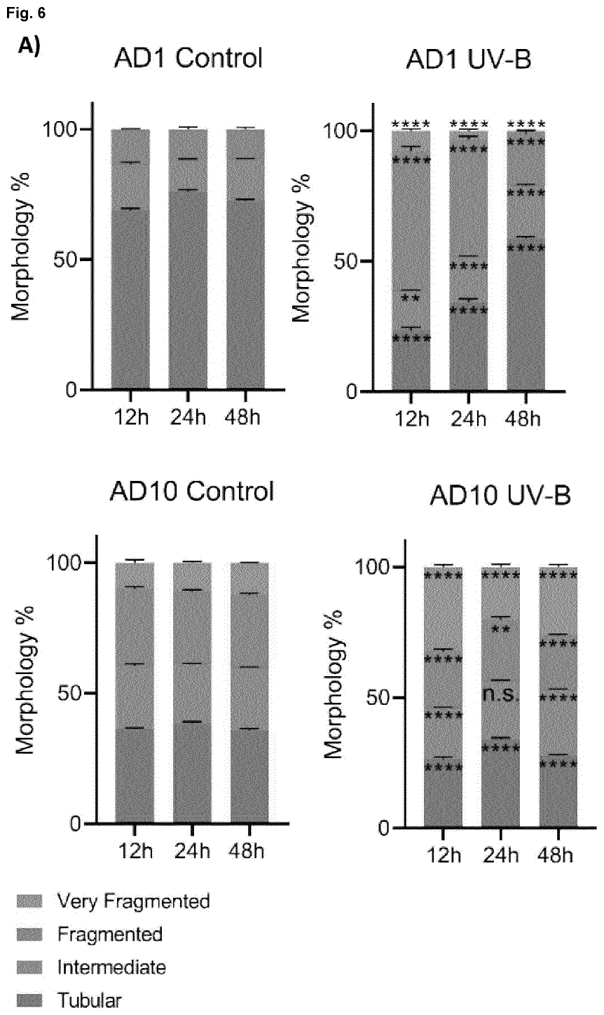

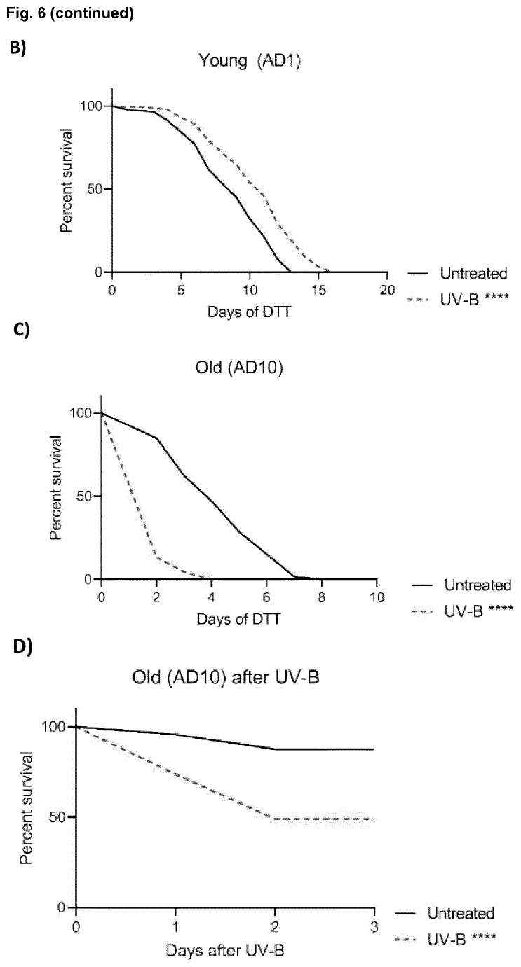

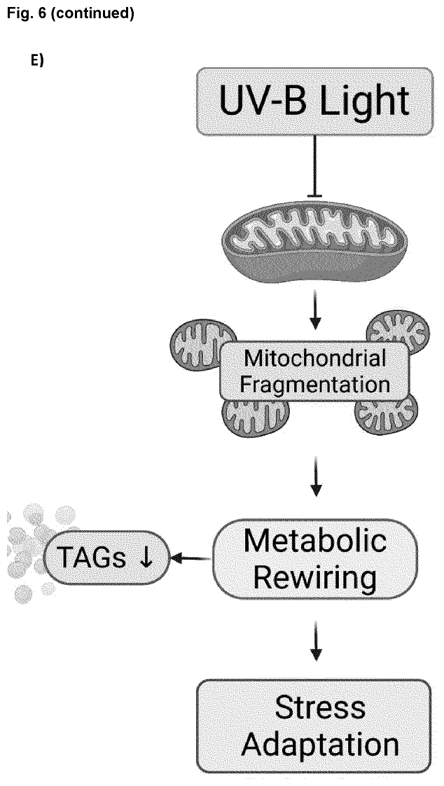

The beneficial effect for a subject being 65 years or younger is that UV-B intervention-related adverse effects are prevented or their risk is reduced. UV-B irradiation can be associated with increased adverse effects in subjects older than 65 years. In these age groups potential disadvantages usually outweigh the potential advantages of the irradiation according to the invention. This effect is also shown in the Example and FIGS. 6A-C, where AD10 age in nematodes is comparable to ≥65 years old in humans.

In embodiments of the invention the narrow-band UV-B radiation induces transient/reversible metabolic remodelling involving one or more of mitochondrial fragmentation/fission, peroxisomal and mitochondrial lipid beta-oxidation, lipid droplet turnover and glycolysis.

Metabolic remodeling can lead in some embodiments to increased metabolic rates in a subject, which can induce increased energy consumption, and might in some cases ultimately lead to weight loss and/or reduction of (excess) fat tissue in a subject. The processes of mitochondrial fragmentation and fission, peroxisomal and mitochondrial lipid beta-oxidation, lipid droplet turnover and glycolysis are involved in the energy metabolism of a cell. Accordingly, in embodiments the method of the invention can induce weight loss by causing metabolic remodeling, which might have an influence on one or more of said processes. In the context of some embodiments of the present invention a transient or reversible effect of the present method on said processes is preferred. Also, the analysis of these processes, their products and/or their enzymes might be used to monitor the efficacy of the present method. Such analysis might be conducted in embodiments by analysis of a sample from the subject, such as a blood, serum, plasma, urine, saliva, sputum, a stool sample, preferably a blood or urine sample.

The method of the present invention can be used for several purposes, in particular for achieving cosmetic benefits, benefits for the personal well-being as well as medical benefits. The purpose of the method may also depend on the subject that undergoes the method of the invention.

In embodiments of the invention the present method is configured for inducing dietary restriction-like metabolic remodelling in the subject.

In some aspects the present invention relates to a cosmetic method for improving the physical appearance of the subject. Embodiments of the present method relating to a cosmetic method are non-therapeutic methods.

In embodiments of the cosmetic method the subject has a body mass index of no more than 25.

In specific embodiments of the invention the subject has a body mass index (BMI) of no more than 30.

A BMI of over 30 is considered indicative of the pathological condition of obesity. The cosmetic method according to the invention preferably relates to the cosmetic treatment of healthy subjects with a BMI below 30, preferably below or equal to 25, wherein the subject desires a weight loss purely to improve their subjective perception of their physical appearance and/or to acquire a leaner physical appearance, but without gaining any health benefits from the weight loss or the cosmetic treatment according to the invention. Accordingly, as embodiments relating to cosmetic methods herein are not concerned with bringing the body of the subject from a pathological state back to a normal, healthy state and/or do not prevent a pathological state they are non-therapeutic methods.

In some aspects the present invention relates to a cosmetic method for weight reduction by inducing metabolic remodeling by means of moderate cosmetic sub-erythemal UV-B exposure of narrow-band UV-B radiation in the range of 305-315 nm.

In embodiments the present cosmetic method induces dietary restriction-like metabolic remodelling in a subject by exposing a skin area of the subject to a sub-erythemal dose of narrow-band UV-B radiation in the range of 305-315 nm.

Such embodiments of the present method relating to a cosmetic method are non-therapeutic methods.

In embodiments of the invention, the cosmetic method is a method for preventing weight gain or the increase of body weight and/or body fat tissue. This cosmetic method is considered to be a non-therapeutic method, not aiming to provide prophylaxis nor a preventative treatment of obesity. The in this context prevented weight gain or increase of body weight and/or body fat tissue is only meant to be within the boundaries of a subjectively perceived physical effect/appearance and/or a subjectively perceived gain in weight and/or fat tissue of a healthy, normal-weight subject with a BMI below 30, preferably below 25. Hence, no health benefits are meant to be acquired through the prevention of weight gain or increase of body weight and/or body fat tissue by the present cosmetic method in the healthy subject with a BMI below 30, preferably below 25.

Accordingly, in embodiments the present cosmetic method enables the prevention of weight gain or induces weight loss in a non-obese subject that desires to keep a lean physical appearance or to gain a leaner physical appearance. In preferred embodiments the present cosmetic method achieves this effect through induction of dietary restriction-like metabolic remodelling. In each embodiment of the present cosmetic method the subject acquires no health benefit from the present cosmetic treatment, but only achieves an individually perceived improvement of its physical appearance. Accordingly, in embodiments of the present cosmetic method the subject acquires no health benefit from weight loss induced by the present cosmetic weight loss. Also, in embodiments of the present cosmetic method the subject acquires no health benefit from metabolic remodelling induced by the present cosmetic method. In embodiments of the present cosmetic method the subject acquires no health benefit from the prevention of weight gain or the prevention of increase of body weight and/or body fat tissue induced by the present cosmetic method. Accordingly, the cosmetic methods described herein are non-therapeutic methods.

In some embodiments it might be of advantage to determine the levels of certain markers in a sample from the subject receiving UV-B radiation according to the invention to estimate an effect of the cosmetic method on the metabolism of the subject, already before weight loss occurs.

In some embodiments a reduction of the levels of one or more markers selected from the group comprising metabolic markers: serum calcium, cholesterol, LDL cholesterol, and triglyceride levels (steady state), serum glucose (both steady state levels and fasting levels), leptin (both steady state levels and fasting levels), insulin (both steady state levels and fasting levels), is indicative of a desired effect of the present cosmetic method on the metabolism of the subject.

In some embodiments an increase of the levels of one or more markers selected from the group comprising adiponectin and HDL cholesterol is indicative of a desired effect of the present cosmetic method on the metabolism of the subject.

Embodiments of the present cosmetic method also have the advantage that they are applicable to non-obese subjects who are willing to acquire a leaner appearance but are not willing to perform and/or are not/less susceptible to dietary approaches. Embodiments of the present cosmetic method are applicable to non-obese subjects that have a limited or no physically ability to perform exercise and/or dietary approaches, but wish to lose weight to gain a leaner physical appearance.

In embodiments the present cosmetic method can also be performed with mobile lamp-systems at home or in a cosmetic studio.

In another aspect the present invention relates to a narrow-band UV-B lamp for use in the cosmetic method according to the present invention, wherein preferably a skin area of the subject is exposed to a sub-erythemal dose of narrow-band UV-B radiation in the range of 305-315 nm emitted from the lamp.

In another aspect the invention relates to a narrow-band UV-B lamp emitting UV-B radiation in the range of 305-315 nm, characterized in that the lamp is configured for whole body irradiation of a human subject.

In embodiments the present invention may also relate to a method of treating obesity and/or metabolic syndrome by inducing metabolic remodeling by means of moderate UV-B exposure. In embodiments the present invention may also relate to a method of treating overweight, obesity and/or metabolic syndrome. In embodiments of the invention the method is a method for treating and/or preventing obesity and/or metabolic syndrome in the subject.

Accordingly, the invention relates in one aspect to a method for treating obesity and/or metabolic syndrome in a subject, the method comprising exposing a skin area of the subject to a sub-erythemal dose of narrow-band UV-B radiation in the range of 305-315 nm.

In embodiments the present method may also provide an effective mimetic for dietary restriction to increase the metabolism and calorie consumption in an individual suffering from obesity, metabolic syndrome and/or requiring weight reduction due to other medical reasons.

The present medical treatment method also has the advantage that it is also applicable to subjects not susceptible to, not willing or not able to use dietary approaches for weight reduction. It is also applicable for subjects that are physically not able or only have a limited ability to perform physical exercise. The present medical treatment method can be applied also in addition to standard therapies to promote, boost or support the effect of dietary approaches and/or physical exercise-based approaches.

In embodiments the present method can also be performed with mobile lamp-systems at home or in a clinic. In embodiments optional regular checkup of blood values can be done by a medical professional.

In another aspect the invention relates to a method for dietary restriction-like metabolic remodelling in a subject comprising exposing a skin area of the subject to a sub-erythemal dose of narrow-band UV-B radiation in the range of 305-315 nm.

In another aspect the present invention relates to a method of treating overweight or obesity in a subject comprising exposing a skin area of the subject to a sub-erythemal dose of narrow-band UV-B radiation in the range of 305-315 nm.

In embodiments the invention relates to a method of treating overweight or obesity in a subject, wherein the subject has a body mass index over 25.

In embodiments the invention relates to a method of treating overweight in a subject, wherein the subject has a body mass index between 25 and 30.

In embodiments the invention relates to a method of treating obesity in a subject, wherein the subject has a body mass index of 30 or higher. In embodiments, the BMI of the subject is over 30, 31, 32, 33, 34, 35, 36, 37, 38, 39, 40, 41, 42, 43, 44, 45, 46, 47, 48, 49, 50 or more.

In one embodiment the invention relates to a method of treating obesity in a subject comprising exposing a skin area of the subject to a sub-erythemal dose of narrow-band UV-B radiation, wherein the initial sub-erythemal dose is determined based on a skin type of the subject.

In embodiments of the present methods of treatment the effects of the UV-B exposure on the metabolism of the treated subject can be assessed by determining blood, plasma and/or serum levels of one or more markers selected from the group comprising metabolic markers, serum calcium, cholesterol, HDL cholesterol, LDL cholesterol, triglyceride levels (steady state), serum glucose, insulin, adiponectin, and leptin (both steady state levels and fasting levels).

In embodiments of the present methods of treatment the irradiation intensity and/or frequency of irradiation is selected to achieve levels of one or more marker indicative of a successful treatment, weight loss and/or metabolic remodelling in the subject, wherein the one or more marker is selected from the group comprising serum calcium, cholesterol, HDL cholesterol, LDL cholesterol, and triglyceride levels (steady state), serum glucose, insulin, adiponectin, leptin (both steady state levels and fasting levels).

In another aspect the present invention relates to a method of treating metabolic syndrome in a subject comprising exposing a skin area of the subject to a sub-erythemal dose of narrow-band UV-B radiation in the range of 305-315 nm.

In embodiments of the method of treating metabolic syndrome the subject has a body mass index (BMI) of over 25, preferably a BMI of at least 30.

In another aspect the invention relates to a narrow-band UV-B lamp for use in the treatment and/or prevention of obesity and/or metabolic syndrome in a subject, wherein the lamp emits UV-B radiation in the range of 305-315 nm and is preferably configured for whole body irradiation of a human subject.

In one embodiment of the narrow-band UV-B lamp for use in the treatment according to the present invention, a skin area of the subject is exposed to a sub-erythemal dose of narrow-band UV-B radiation in the range of 305-315 nm emitted from the lamp.

In another aspect the invention relates to a narrow-band UV-B lamp emitting UV-B radiation in the range of 305-315 nm, characterized in that the lamp is configured for whole body irradiation of a human subject.

In another aspect the invention relates to UV-B radiation for use in the treatment and/or prevention of obesity and/or metabolic syndrome in a subject, wherein the UV-B radiation is in the range of 305-315 nm and is preferably configured for whole body radiation of a human subject.

Embodiments and features of the invention described with respect to the methods of the present invention, in particular the cosmetic method or the method of treatment, the UV-B lamp and the UV-B radiation for use according to the invention, and the various other aspects of the invention described herein, are considered to be disclosed with respect to each and every other aspect of the disclosure, such that features characterizing the methods, may be employed to characterize the UV-B radiation for use according to the invention or the lamp and vice-versa. The various aspects of the invention are unified by, benefit from, are based on and/or are linked by the common and surprising finding of the beneficial and optionally curative effect of the present method comprising exposing a skin area of the subject to a sub-erythemal dose of narrow-band UV-B radiation in the range of 305-315 nm.

### DETAILED DESCRIPTION OF THE INVENTION

All cited documents of the patent and non-patent literature are hereby incorporated by reference in their entirety.

The present invention is directed to a method for reducing body weight of a subject comprising exposing a skin area of the subject to a sub-erythemal dose of narrow-band UV-B radiation in the range of 305-315 nm, wherein preferably the narrow-band UV-B radiation induces transient/reversible metabolic remodeling and wherein the method is preferably a cosmetic method. The invention further relates to methods of treating obesity, overweight and/or metabolic syndrome as well as to a narrow-band UV-B lamp and UV-B radiation both for use in the treatment and/or prevention of obesity and/or metabolic syndrome in a subject.

The term “subject” includes a mammalian, an animal, a human, preferably a human.

The present invention relates in one embodiment to a cosmetic method for reducing body weight of a subject wherein one of the effects of this method leading to weight loss is the remodeling of metabolism.

Herein a “sample” may be a biological sample that is obtained or isolated from the subject. “Sample” as used herein may, in some embodiments e.g., refer to a sample of bodily fluid or excrements obtained for the purpose of analysis, prognosis, or evaluation of the effects achieved or caused in a subject that was exposed to sub-erythemal UV-B radiation according to the invention. Preferably herein, the sample is a sample of a bodily fluid, such as blood, serum, plasma, cerebrospinal fluid, urine, saliva, sputum, pleural effusions, cells, a cellular extract, a tissue sample, a tissue biopsy, a stool sample and the like. Particularly, the sample is blood, blood plasma, blood serum, or urine.

The medical definition of “metabolism” generally relates to the sum of the physical and chemical processes by which living organized substance is built up and maintained (anabolism), and by which large molecules are broken down into smaller molecules to provide energy to an organism (catabolism). Essentially these processes are concerned with the disposition of the nutrients absorbed into the blood following digestion. Metabolism involves the consumption of fuel (glucose and fatty acids), the production of heat and the utilization of many constructional and other biochemical elements provided in the diet, such as amino acids, fatty acids, carbohydrates, vitamins, minerals and trace elements. The basal metabolic rate refers to the lowest rate obtained while an individual is at complete physical and mental rest. Metabolic rate usually is expressed in terms of the amount of heat liberated during the chemical reactions of metabolism. Basal metabolism describes usually the minimal energy expended for the maintenance of respiration, circulation, peristalsis, muscle tonus, body temperature, glandular activity, and the other vegetative functions of the body.

“Mitochondria” are double-membrane-bound organelles found in cells of most eukaryotic organisms. Mitochondria play many important roles for normal cellular function, wherein the probably most important one is the generation of adenosine triphosphate (ATP), the energy molecule for cellular processes, through oxidative phosphorylation. Mitochondria metabolize lipids and sugar through fatty acid β-oxidation and oxidative phosphorylation to generate ATP, wherein fatty acid β-oxidation and oxidative phosphorylation are tightly linked biochemically. At the same time the ATP generated in mitochondrial metabolism is used as energy source in pathways that produce the building blocks necessary for macromolecule synthesis, such as lipid metabolism. Mitochondrial metabolism is the main function of mitochondria, which can be activated or reduced. This depends on the energy demand of cells/organism as well as on food availability. As used herein, an increase in mitochondrial metabolism and an increased mitochondrial function in particular refer to an increased rate of mitochondrial respiration/oxidative phosphorylation. If mitochondrial function increases, it means that mitochondrial metabolism becomes more active and more efficient and ATP production is increased, while resources from food are consumed.

Mitochondria are the generators of most of a cell's supply of adenosine triphosphate (ATP; a source of chemical energy for a cell). Mitochondria are distributed throughout the entire cell and behave as an interconnected network while simultaneously maintaining contact with other organelles. This cell-wide distribution of mitochondria is conducive for responding to perturbations that require global responses such as increased energy production. “Mitochondrial fragmentation” and “mitochondrial fission” enable the organelles to behave as isolated organelles contrary to an interconnected network, which required fusion. These morphological changes are closely related to mitochondrial function, including regulation of metabolism. Mitochondrial fusion is particularly important in respiratory active cells and is required for maximum respiratory capacity. The fusion allows the distribution of metabolites, enzymes, and mitochondrial gene products throughout the entire mitochondrial compartment. Mitochondrial fragmentation is usually found in resting cells and mitochondrial fission plays a role in degradation of dysfunctional organelles.

“Dietary restriction-like metabolic remodeling” describes in embodiments a process comprising upregulation of glycolysis, downregulation of cellular lipid droplet components and upregulation of beta oxidation enzymes both mitochondrial and peroxisomal. In embodiments the metabolic remodeling is transient and/or reversible and involves at least one of the processes of mitochondrial fragmentation and/or fission, peroxisomal and mitochondrial lipid beta-oxidation, lipid droplet turnover and glycolysis. In preferred embodiments this metabolic remodeling leads to results comparable to effects of dietary restriction, namely one or more of the effects selected from weight loss, reduction of body fat, reduction of blood glucose levels, reduction of blood LDL levels, increase in blood HDL levels and/or reduction of blood triglycerides. In embodiments the dietary restriction-like effects of metabolic remodeling induced by methods according to the invention result in the same benefits for health and/or wellbeing of a subject as dietary restriction and/or reduced calorie intake.

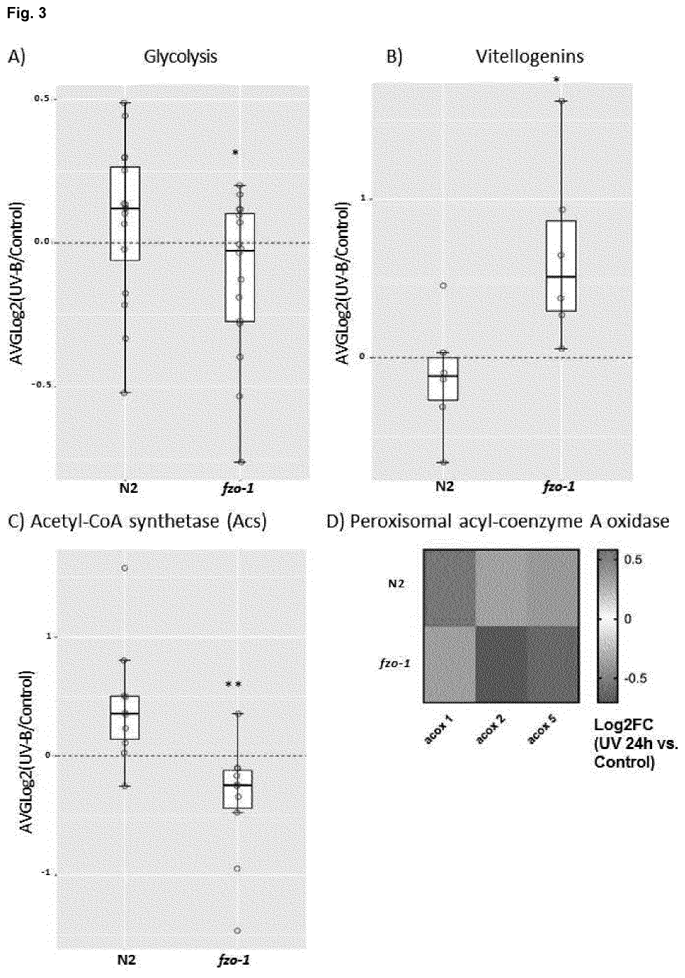

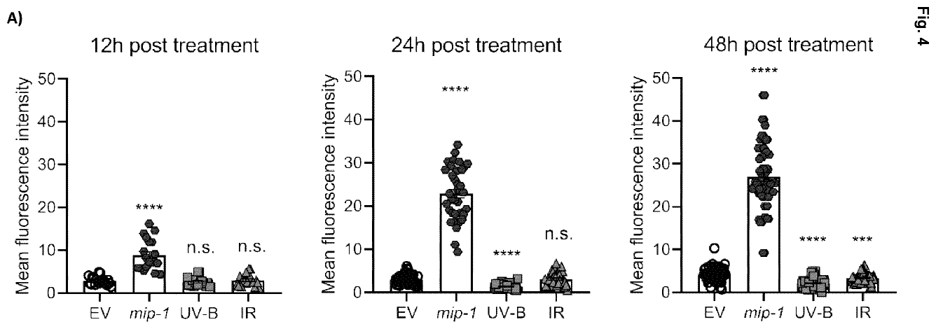

An example of embodiments of dietary restriction-like metabolic remodeling in the model organism C. elegans can be derived from the Example and FIG. 3. In this figure, the relative expression of proteins implicated in glycolysis (panel A), lipid droplet formation (panel B), mitochondrial lipid β-oxidation (panel C) and peroxisomal lipid β-oxidation (panel D) was assessed by proteomics in wild type (N2) and mitochondrial fusion deficient (fzo-1 mutant) animals 24 h after UV-B treatment. The data show upregulation of glycolysis and β-oxidation components as well as downregulation of lipid droplet proteins in wild type subjects following UV-B exposure, consistent with the changes of these pathways observed in response to DR. These changes are consistent with the enhanced catabolism of sugars and fats by the animals. The data also show that interference with mitochondrial plasticity (fzo-1 mutation) abrogates beneficial effects of UV-B on metabolic remodeling.

Metabolic remodeling can be assessed in embodiments by measuring responses of cells or organisms to, for example, changes in nutrient availability or exposure to other stimuli, such as UV-B radiation according to the invention. For example, in embodiments the body weight of a subject can be monitored in response to the UV-B irradiation according to the present invention. Also, in embodiments the oxygen consumption and/or the extracellular acidification rate of cells can be assessed in order to determine the contribution of mitochondrial respiration/oxidative phosphorylation and glycolysis to the metabolism of a cell. This can be done by using the so-called Seahorse-technology of Agilent or other techniques known to the skilled person for measuring oxidative phosphorylation in cells and the rate or ratio of the respective pathways.

Herein the terms “marker” and “parameter” may be used interchangeably.

Herein the term “blood” or “blood sample” may be used as a generic term for and/or may comprise whole blood, plasma and/or serum and/or other blood components. Accordingly, the term “blood sample”, “blood value” or “blood level” can also comprise plasma samples, values and levels as well as serum samples, values and levels.

A “marker” or “parameter” may herein refer to certain molecules and their levels in a sample obtained from a subject. In some embodiments markers may be, without being limited to, serum calcium, cholesterol, HDL cholesterol, LDL cholesterol, and triglyceride levels, serum glucose, insulin, adiponectin, leptin, blood NLR and serum interleukin (IL)-6, CRP, and/or IL-1β.

“LDL” or “Low-density lipoprotein cholesterol” is one of the five major groups of lipoprotein which transport fat molecules around the body in the extracellular fluid and deliver them to the cells. LDL is the type of cholesterol that can eventually build up within the walls of arteries, leading to a heart attack or stroke. This is why LDL is often referred to as “bad” cholesterol.

“HDL” or “High-density lipoprotein cholesterol” particles remove fat molecules from cells, unlike the larger lipoprotein particles, which deliver fat molecules to cells. Increasing concentrations of HDL particles are associated with decreasing accumulation of atherosclerosis within the walls of arteries, reducing the risk of sudden plaque ruptures, cardiovascular disease, stroke and other vascular diseases. Hence, HDL is often referred to as “good” cholesterol, as it picks up excess cholesterol in the blood and transports it back to the liver, where it is broken down and removed from the body.

Normal or healthy levels of cholesterol are different, depending on age and sex: In people 19 and younger LDL cholesterol levels of less than 110 milligrams and HDL levels of more than 45 milligrams are considered healthy. In men 20 and older LDL cholesterol values of less than 100 milligrams and HDL values of more than 40 milligrams are considered healthy. In women 20 and older LDL cholesterol values of less than 100 milligrams and HDL values of more than 50 milligrams are considered healthy.

“Leptin” is a hormone that is produced almost exclusively by fat cells. Leptin conveys a feeling of satiety via feedback to the nervous system. Leptin also regulates glucose homeostasis (glucose-lowering effect) regardless of body weight. This effect can presumably be attributed to the improvement in insulin sensitivity in muscle tissue and the liver. Leptin has a positive correlation with body fat-leptin therefore reflects the body fat content.

“Adiponectin” is a polypeptide of 244 amino acids with a collagen-like structure and is only produced in adipocytes (fat tissue cells). It works via 2 receptors, AdipoR1 (in the skeletal muscle) and AdipoR2 (in the liver). Low fat reserves lead to an increased formation of adiponectin, whereas full stores lead to a reduced formation. Adiponectin reduces free fatty acids in the blood and improves insulin sensitivity in fat cells, the liver and skeletal muscle. Furthermore, vasoprotective and anti-inflammatory effects of adiponectin have been described. Hence, the adiponectin level is negatively correlated (decreased in the case of) with obesity, insulin resistance, type 2 diabetes, hypertension, high fasting glucose, increased LDL and total cholesterol. The adiponectin level, on the other hand, is positively correlated with the level of HDL cholesterol.

“Interleukin 6” (IL-6) is an interleukin that acts as both a pro-inflammatory cytokine and an anti-inflammatory myokine. In inflammation IL-6 is responsible for stimulating acute phase protein synthesis, as well as the production of neutrophils in the bone marrow. It supports the growth of B cells and is antagonistic to regulatory T cells. Increased serum and/or blood (IL)-6 levels can be indicative of an excessive inflammatory reaction.

“C-reactive protein” (CRP) is a ring-shaped pentameric protein found in blood plasma, whose circulating concentrations rise in response to inflammation. It is an acute-phase protein of hepatic origin that increases following interleukin-6 secretion by macrophages and T cells.

“Interleukin 1 beta” (IL-1β) is a cytokine produced by macrophages and is an important mediator of the inflammatory response, and is involved in a variety of cellular activities, including cell proliferation, differentiation, and apoptosis. Increased levels of IL-1 β in blood and/or plasma are indicative of increased inflammation.

“Neutrophil lymphocyte ratio” (NLR) is a parameter for systemic inflammation. It is calculated by dividing the number of neutrophils by number of lymphocytes, usually from peripheral blood sample. Higher NLR in blood and/or plasma is an independent predictor of mortality in cardiovascular disease patients and a predictor for poor prognosis in cancer patients.

“Lipid beta-oxidation” occurs in both mitochondria and peroxisomes. Mitochondria catalyze the beta-oxidation of the bulk of short-, medium-, and long-chain fatty acids derived from food, which constitutes the major process by which fatty acids are oxidized to generate energy. Peroxisomes contribute in beta-oxidation chain shortening of long-chain and very-long-chain fatty acyl-coenzyme (CoAs), long-chain dicarboxylyl-CoAs, the CoA esters of eicosanoids, 2-methyl-branched fatty acyl-CoAs, and the CoA esters of the bile acid intermediates di- and trihydroxycoprostanoic acids, wherein H₂O₂ is generated (Reddy JK and Hashimoto T, Annu Rev Nutr., 2001).

“Lipid droplet turnover” describes the consumption of lipid droplets, which are dynamic lipid-storage organelles of a cell that are formed when there is a constant exogenous supply of fatty acids. When metabolic conditions change, the fats stored in lipid droplets can be mobilized for the metabolic process of lipolysis whereby they contribute to a cell's energy homeostasis.

“Glycolysis” describes the metabolic process wherein glucose is converted into pyruvic acid. The energy obtained from this process is stored in ATP and reduced nicotinamide adenine dinucleotide (NADH). Under aerobic conditions, pyruvate can diffuse into mitochondria, where it enters the citric acid cycle and is oxidized to carbon dioxide and water by mitochondrial enzymes.

Herein “proteostasis” describes a functional cellular network comprising molecular chaperones and proteolytic machineries and their regulators, wherein these factors coordinate protein synthesis with polypeptide folding, the conservation of protein conformation and protein degradation, namely a balanced and functional proteome.

“Ultraviolet radiation” (“UV” radiation) generally refers to a form of electromagnetic radiation with a wavelength between 100 nm and 400 nm which is, for example, emitted by the sun or artificial sources, such as UV-radiation emitting lamps. UV radiation can be divided into three groups, namely UV-A, UV-B and UV-C, wherein UV-A is generally referring to wavelengths between 400-315 nm, UV-B to wavelengths between 315-280 nm and UV-C to wavelengths between 280-100 nm. In the context of embodiments of the present invention, when referring to UV-A, UV-B or UV-C, respective nanometer wavelength ranges with up to +/−10%, such as 9, 8, 7, 6, 5, 4, 3, 2, 1%, are meant. UV-A rays have the least energy among UV rays and are associated with long-term skin damage, skin aging and certain indirect damage to cellular DNA. Which might contribute to skin cancer. UV-B rays have more energy than UV-A rays, wherefore they can induce DNA-damage in skin cells directly, leading to short term effects, such as sunburn, and long-term effects, such as skin cancer. UV-C rays have the most energy of all UV rays. UV-C rays emitted by the sun react with ozone high in our atmosphere and generally don't reach the earth's surface.

Sunburn is a form of radiation burn that affects living tissue, such as skin, and results from an overexposure to ultraviolet (UV) radiation, commonly from the sun or artificial UV sources. Common symptoms in humans and other animals include red or reddish skin that is hot to the touch, pain, general fatigue, and mild dizziness. Excessive UV radiation is the leading cause of primarily non-malignant skin tumors. Moderate sun tanning without burning can also prevent subsequent sunburn, as it increases the amount of melanin, a photoprotective pigment that is the skin's natural defense against overexposure.

A “sub-erythemal dose” refers in the context of the present invention to a dose of irradiation, preferably UV-irradiation, that does not cause any redness, injury and/or long-term inflammation of the skin. “Erythema” describes the condition of skin redness caused by dilatation and congestion of the capillaries, which can be a sign of skin injury, inflammation and/or infection.

A “narrow-band UV-B radiation” (NB-UV-B) refers to a subrange of the UV-B radiation spectrum. Narrow-band UV-B radiation may herein refer to radiation with a wavelength between 300 and 315 nm. In preferred embodiments the narrow-band UV-B radiation is a radiation with a wavelength between 305 and 315 nm. In some embodiments the narrow-band UV-B radiation has a wavelength between 311-312 nm. In some embodiments the narrow-band UV-B radiation has a wavelength of 312 nm. In other embodiments the narrow-band UV-B radiation has a wavelength of 311 nm. In the context of embodiments of the present invention, when referring to narrow-band UV-B radiation respective nanometer wavelength, ranges with up to +/−10%, such as 9, 8, 7, 6, 5, 4, 3, 2, 1%, are meant.

The “Fitzpatrick scale” or “Fitzpatrick skin type” or “Fitzpatrick phototype” is a numerical classification schema for human skin color. It was developed to measure the correct dose of UVA for PUVA therapy (Psoralen and UVA-light therapy) and is a standard dermatological classification tool for human skin pigmentation. The Fitzpatrick skin types can be classified by their reactions to UV-exposure as follows: Type I always burns, never tans; Type II usually burns, tans minimally (light colored but darker than fair); Type III experiences sometimes mild burn, tans uniformly (golden honey or olive); Type IV burns minimally, always tans well (moderate brown); Type V very rarely burns, tans very easily (dark brown) and Type VI never burns (deeply pigmented dark brown to darkest brown).

“Exposure to UV-B radiation” describes herein the exposure of a subject or irradiation of a subject with UV radiation, preferably with majorly or exclusively UV-B radiation. In preferred embodiments the UV radiation is originating from an artificial source. The artificial source may be a light bulb, a LED and/or a lamp emitting, in case of UV-B radiation, electromagnetic radiation of a wavelength between 280-315 nm, preferably between 300 and 315 nm, more preferably between 305 and 315 nm. In some embodiments the artificial source is emitting electromagnetic radiation of a wavelength between 311-312 nm. In the context of embodiments of the present invention, when referring to the electromagnetic radiation emitted by the artificial source nanometer wavelength ranges with up to +/−10%, such as 9, 8, 7, 6, 5, 4, 3, 2, 1%, are meant.

The “reduction of body weight” may refer in preferred embodiments of the present invention to the loss of excess fat tissue or excess fat depositions on a subject's body.

In some embodiments the excess fat tissue might have a negative effect on the health and/or wellbeing of the subject. In such embodiments not just the physical appearance of the subject can be modified by the method according to the invention but also the health and/or wellbeing of the subject can be improved and, optionally, symptoms and/or downstream effects of the excess body fat can be prevented, reduced or even abolished. The present method achieves this surprising effect by inducing a remodeling of the metabolism of the subject and/or by increasing the metabolism, the fat metabolism and/or the calorie turnover of the subject.

In some embodiments the excess fat might have a (subjective) negative effect on the physical appearance and/or wellbeing of the subject and/or might lead or contribute to an undesired appearance of a subject's body. The present method aims in some embodiments to improve the physical appearance of the subject. This means that in some embodiments the physical appearance of the subject is changed to a leaner appearance, e.g. by weight loss and/or by reduction of excess body fat tissue of the subject. In some embodiments this means that the body shape of the subject is modified by weight loss and/or by reduction of excess body fat tissue in specific areas of the body or in all areas of the body. In some embodiment such method may be a cosmetic method. In embodiments where the subject is unsatisfied with its physical appearance, the reduction of body weight according to the present invention can also increase the (subjective) wellbeing of the subject.

A “body mass index” (BMI) describes a value calculated from a person's weight in kilograms divided by the square of height in meters. A BMI between 18.5 and 25 is considered to be healthy or normal. A BMI over 25 can indicate overweight or obesity. BMI weight categories under 18.5 or over 25 may lead to health problems, but do not diagnose the health of an individual per se.

“Overweight” and “obesity” are defined as medical conditions in which abnormal or excessive body fat accumulation presents a risk to the health of a subject. Obesity is defined by body mass index (BMI) and further evaluated in terms of fat distribution via the waist-hip ratio and total cardiovascular risk factors. A body mass index (BMI) over 25 is considered overweight, and over 30 is regarded obese. Obesity is considered one of the leading preventable causes of death worldwide and increases the risk of many physical and mental conditions. These comorbidities are most commonly shown in “metabolic syndrome”, which describes a combination of at least three of the following five medical disorders comprising: abdominal obesity, high blood pressure, high blood sugar, high serum triglycerides, and low serum high-density lipoprotein (HDL). The syndrome is thought to be caused, at least partially, by an underlying disorder of energy utilization and storage. Metabolic syndrome increases the risk of heart disease, stroke and type 2 diabetes. The continuous provision of energy via dietary carbohydrate, lipid, and protein fuels, unmatched by physical activity/energy demand creates a backlog of the products of mitochondrial oxidation, a process associated with progressive mitochondrial dysfunction and insulin resistance.

In one embodiment the present invention is directed to the treatment and/or prevention of overweight, obesity and/or metabolic syndrome in a subject.

As used herein, “treatment” or “therapy” generally means to obtain a desired pharmacological effect and/or physiological effect. The effect may be prophylactic in view of completely or partially preventing a disease and/or a symptom, for example by reducing the risk of a subject having a particular disease or symptom, or may be therapeutic in view of partially or completely curing a disease and/or adverse effect of the disease. In the present invention, “therapy” includes arbitrary treatments of diseases or conditions in mammals, in particular, humans, for example, the following treatments (a) to (c): (a) Prevention of onset of a disease, condition or symptom in a patient; (b) Inhibition of a symptom of a condition, that is, prevention of progression of the symptom; (c) Amelioration of a symptom of a condition, that is, induction of regression of the disease or symptom.

Herein “whole body irradiation of a human subject” means irradiation of at least 5%, 10%, 15%, 20%, 25%, 30%, 35%, 40%, 45%, 50%, 55%, 60%, 65%, 70%, 75%, 80%, 85%, 90%, 95%, 99% or at least 99.5% or of 100% of the body surface of a subject. In preferred embodiments of whole-body irradiation at least 25% of the body surface of a subject are irradiated. Preferably, the irradiation is UV irradiation, even more preferably UV-B irradiation.

The instant disclosure also includes kits, packages and multi-container units containing the herein described lamps and/or means for administering the UV-B radiation for use in the prevention and/or treatment of diseases and other conditions in subjects, as described herein.

### FIGURES

The invention is further described by the following figures. These are not intended to limit the scope of the invention but represent preferred embodiments of aspects of the invention provided for greater illustration of the invention described herein.

### BRIEF DESCRIPTION OF THE FIGURES

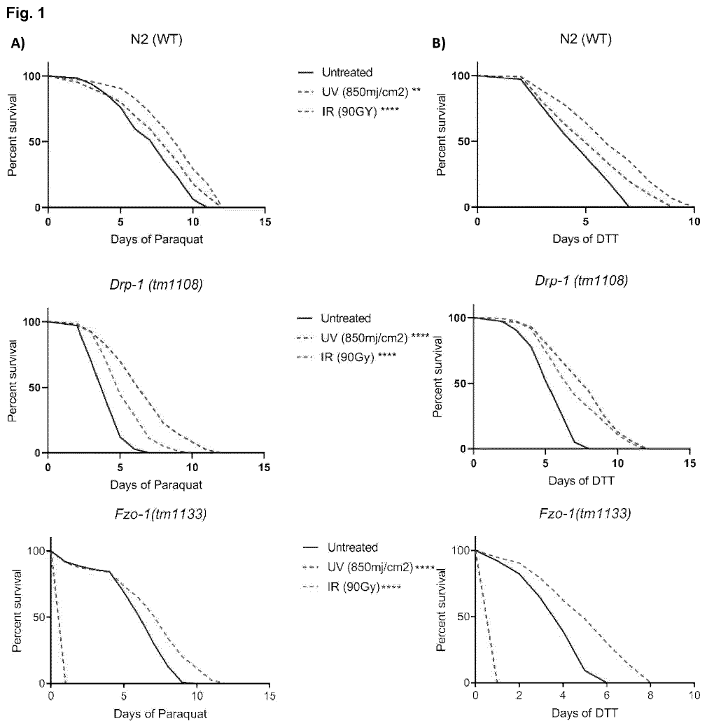

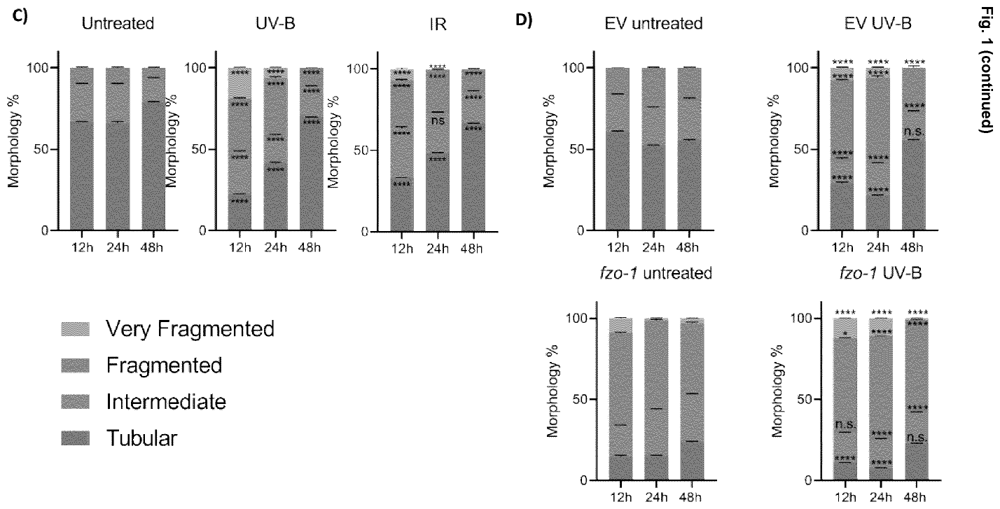

FIG. 1: Mitochondrial fusion is required for adaptive stress responses to UV-B.

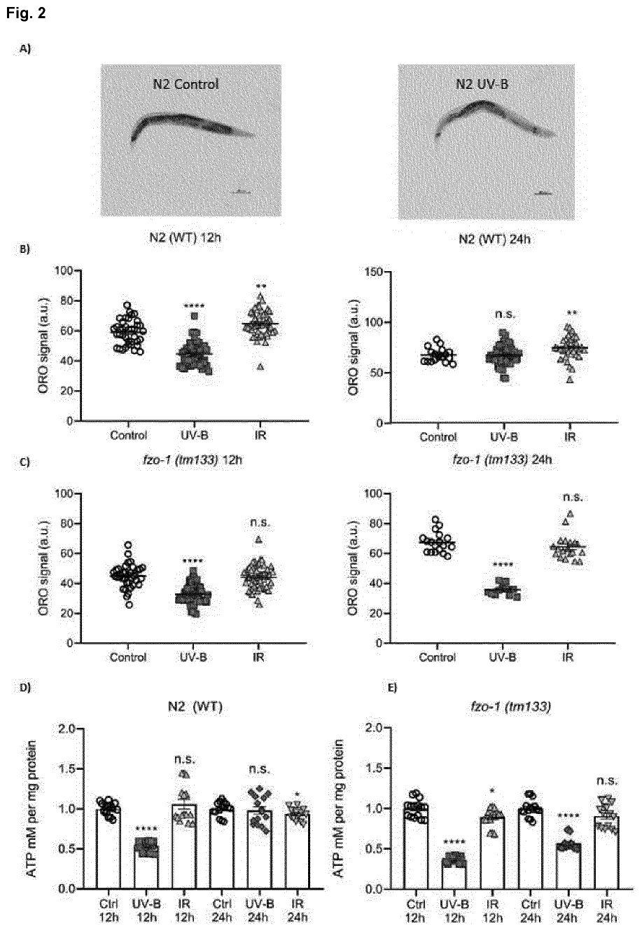

FIG. 2: UV-B triggers transient dietary restriction-like changes of energy metabolism.

FIG. 3: Metabolic remodeling responses to UV-B are reversed by the lack of mitochondrial fusion.

FIG. 4: Moderate UV-B exposure does not induce lasting mitochondrial damage in wild type animals.

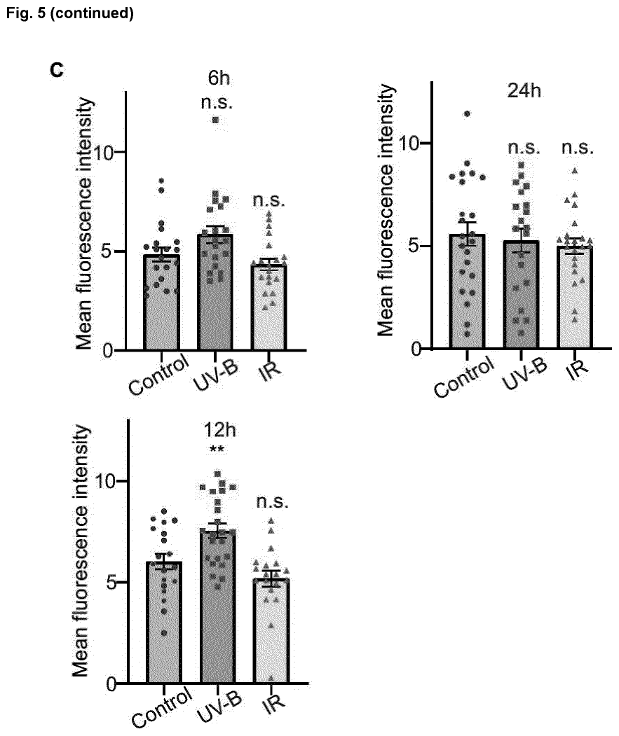

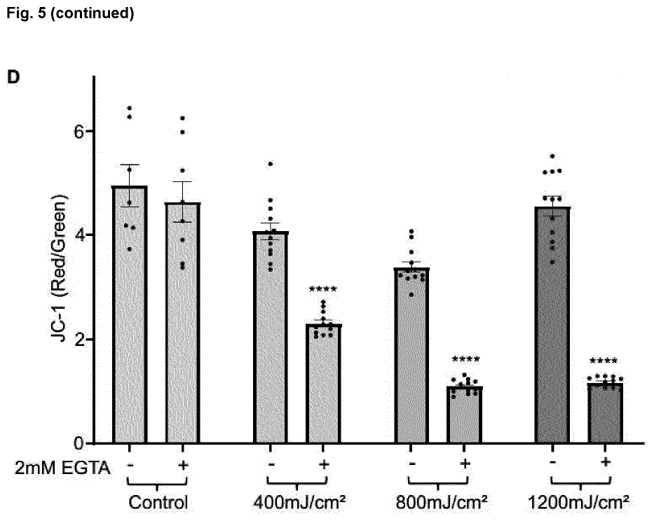

FIG. 5: Adaptive UV effects involve AY distortion, mitochondrial biogenesis and Ca2+ signaling.

FIG. 6: Aging-associated decline of mitochondrial fusion abrogates UV-B benefits in late life and confers UV-B toxicity.

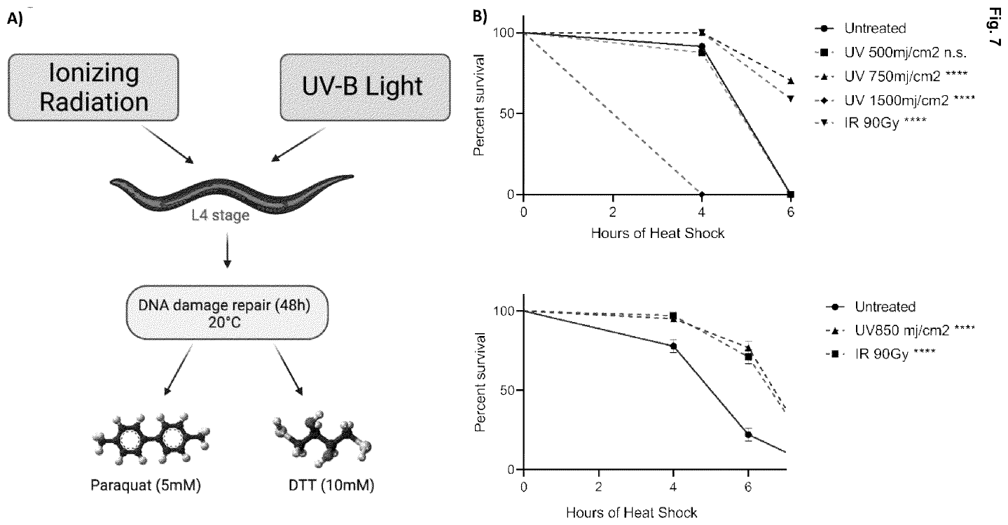

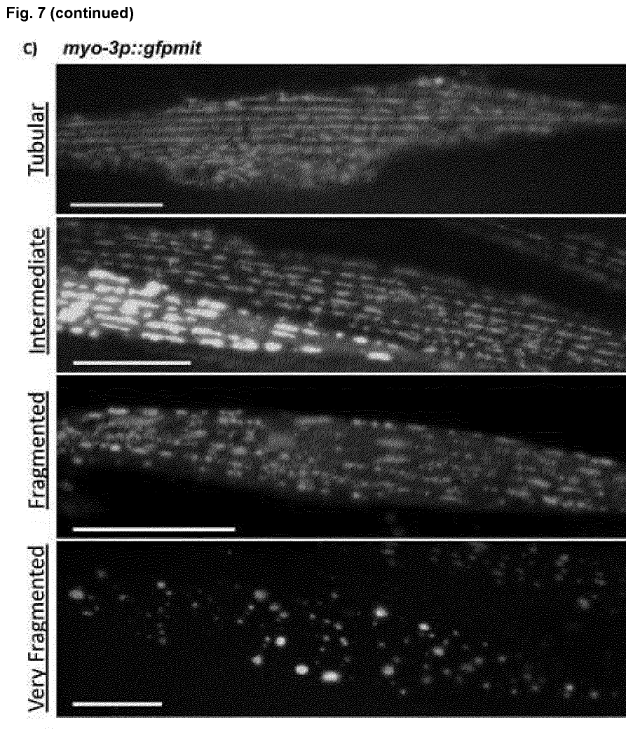

FIG. 7: Assessment of UV-B and IR effects on stress responses and mitochondrial morphology.

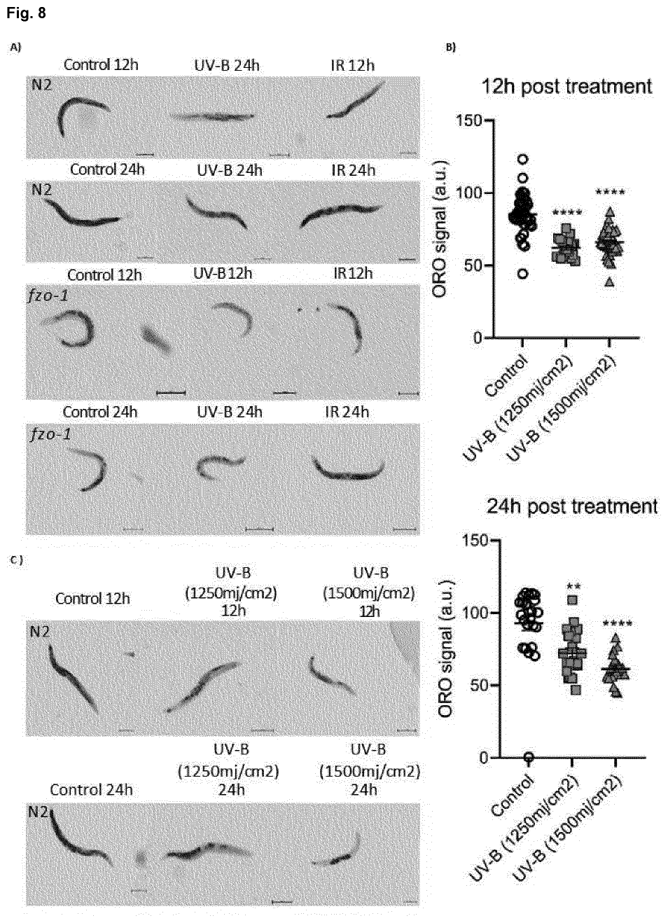

FIG. 8: UV-B treatment promotes a reduction of lipid content in a dose dependent manner.

FIG. 9: Immune and proteostasis responses to UV-B are independent of mitochondrial fusion capacity.

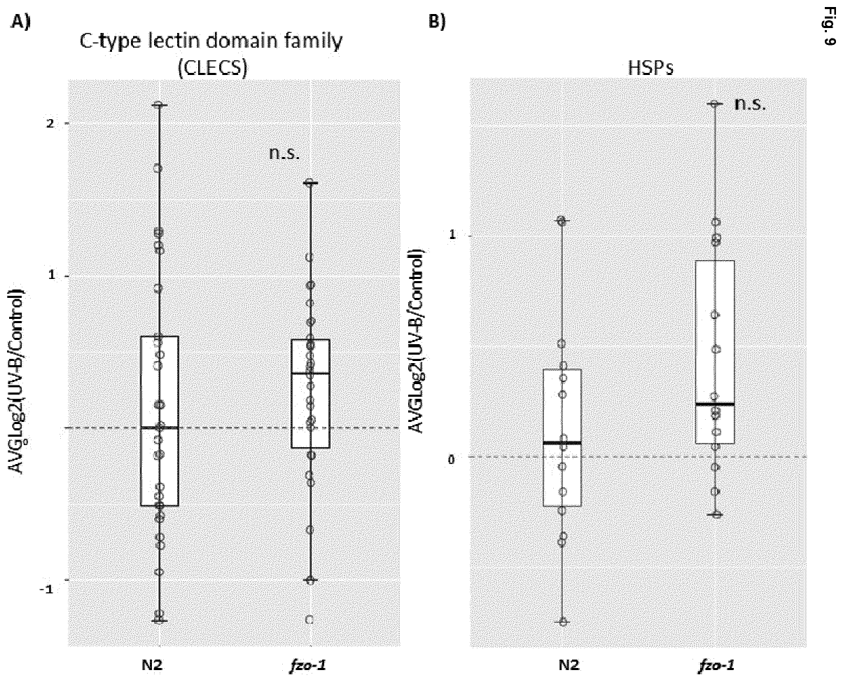

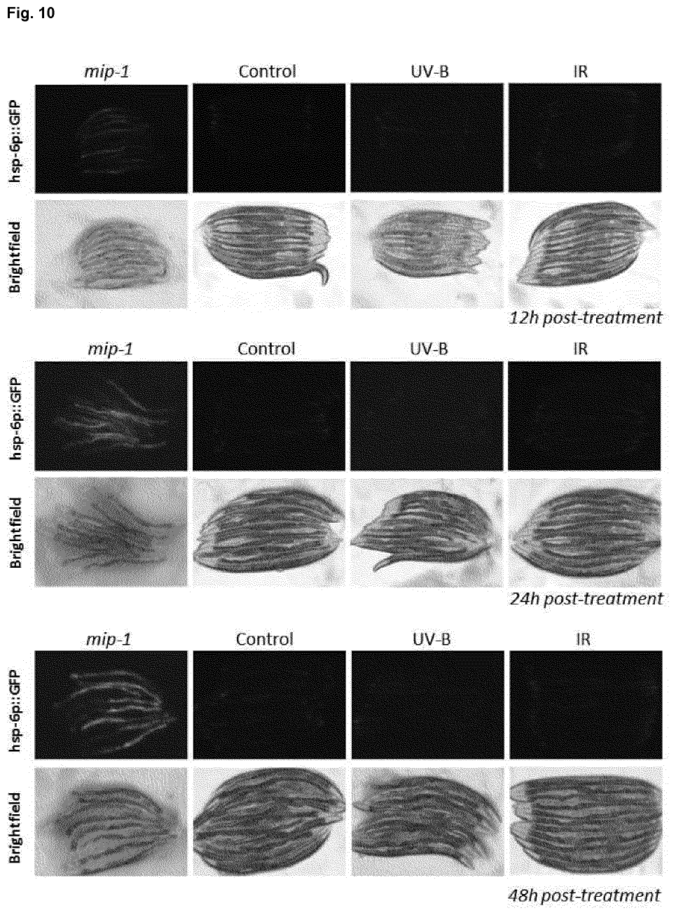

FIG. 10: Moderate UV-B exposure does not trigger mitochondrial UPR.

FIG. 11: Moderate UV-B exposure induces the ER unfolded protein response.

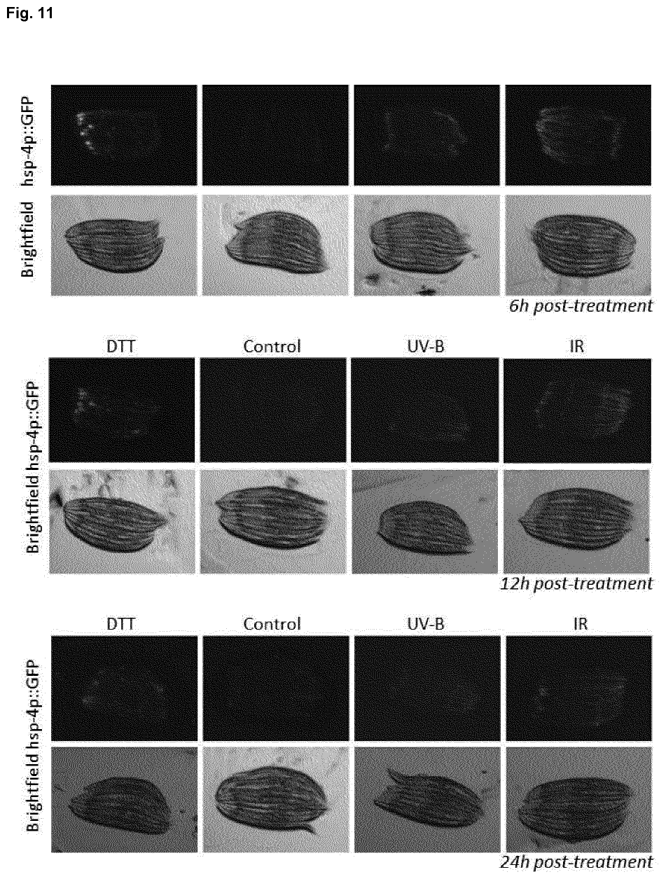

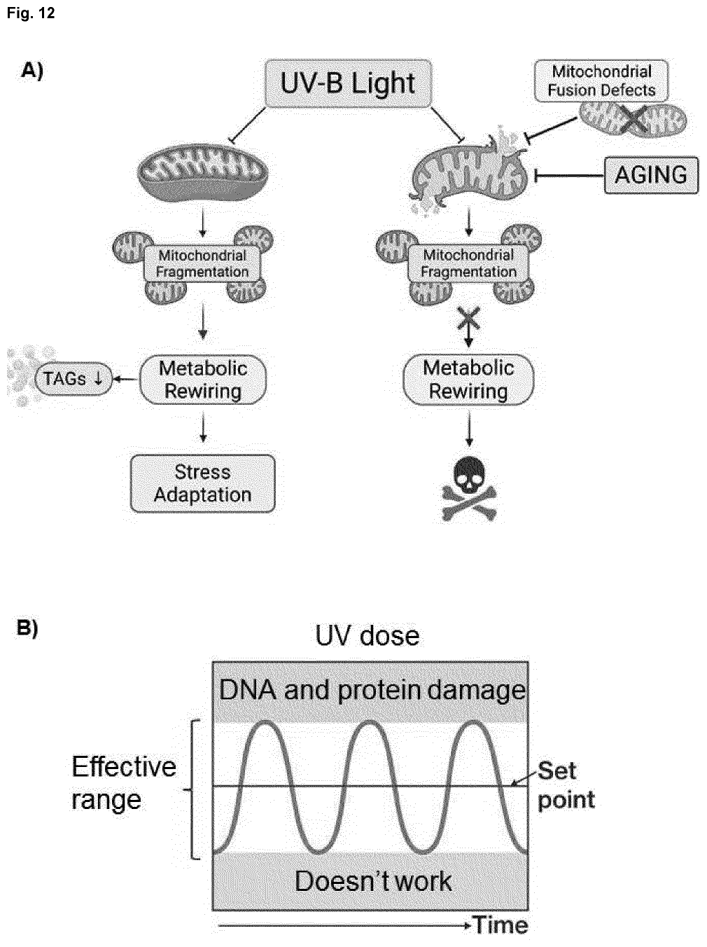

FIG. 12: Mitochondrial fusion defects abrogate metabolic benefits of UV-B treatment in late life.

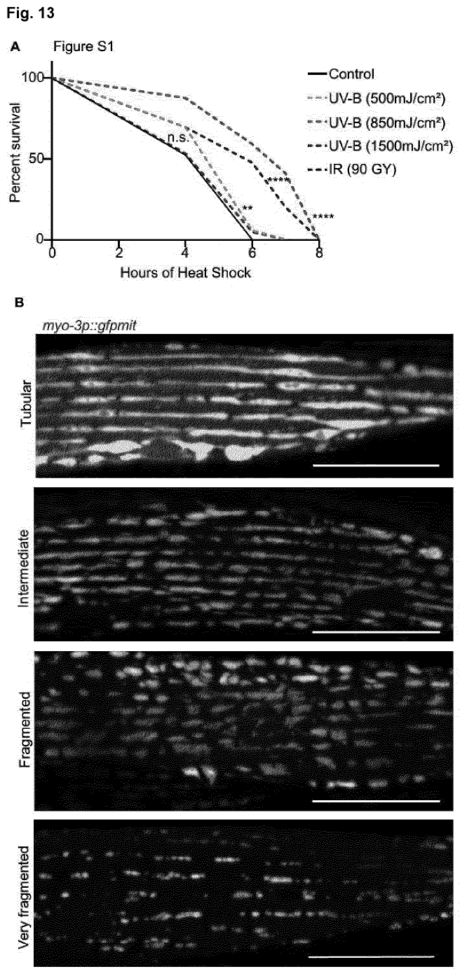

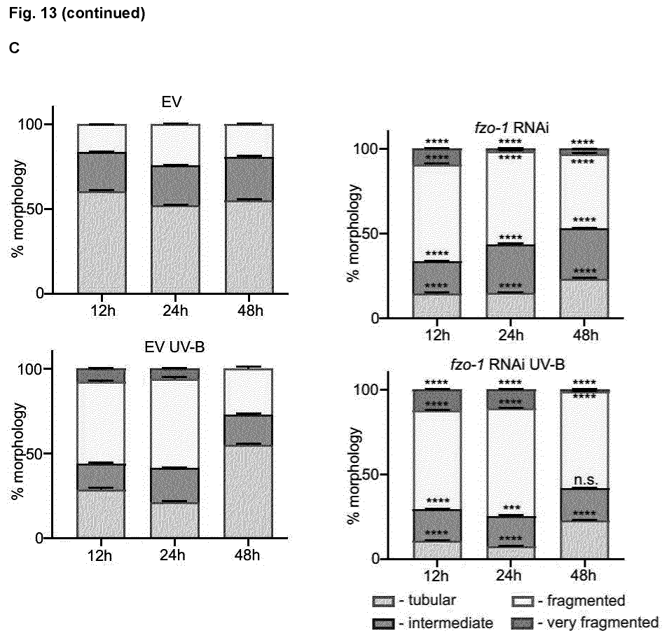

FIG. 13: UV-induced mitochondrial fragmentation is exacerbated by mitofusin gene knock down.

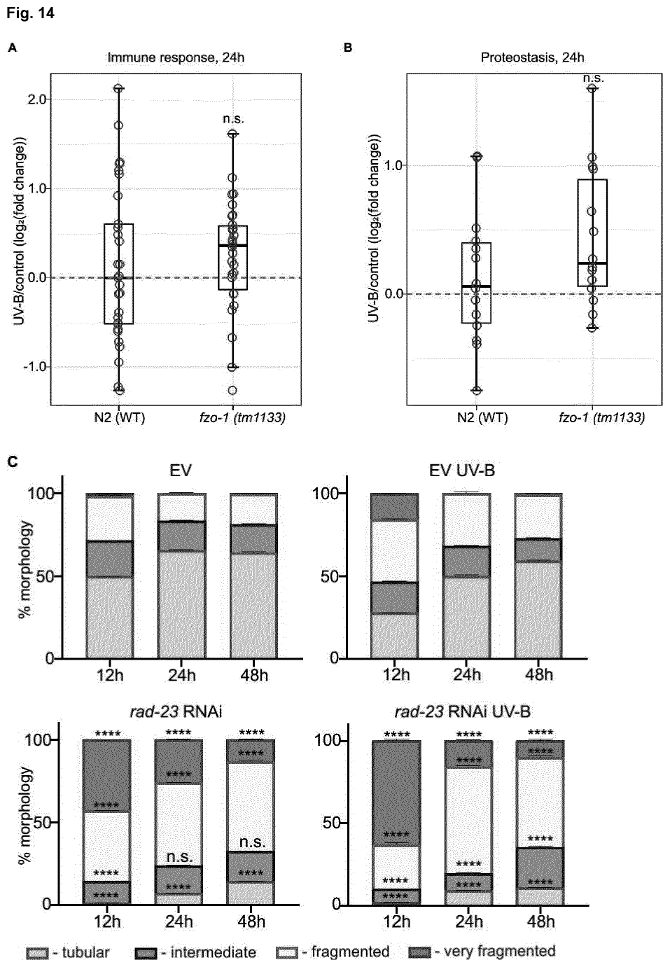

FIG. 14: Mitochondrial and nuclear effects of UV-B are independent.

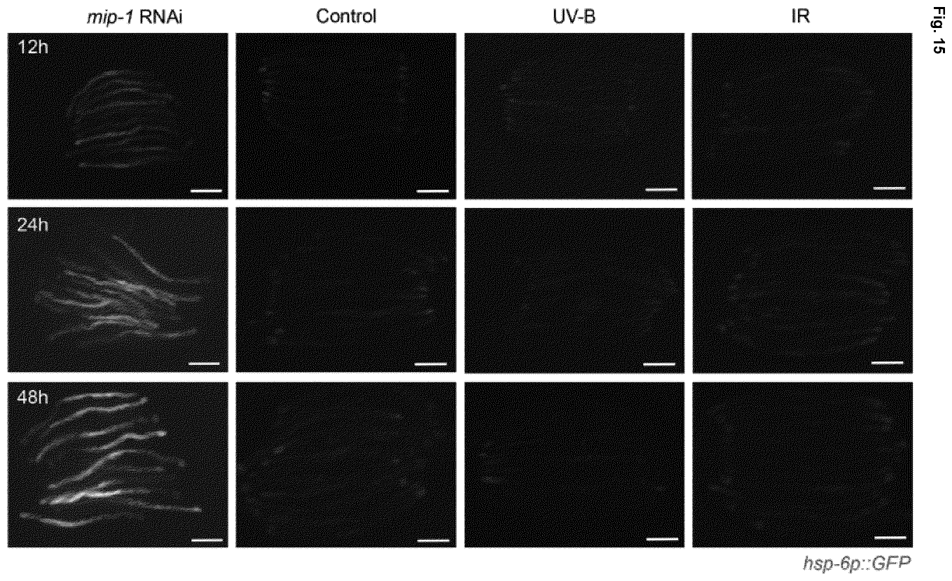

FIG. 15: Moderate UV and IR treatments do not activate mitochondrial UPR.

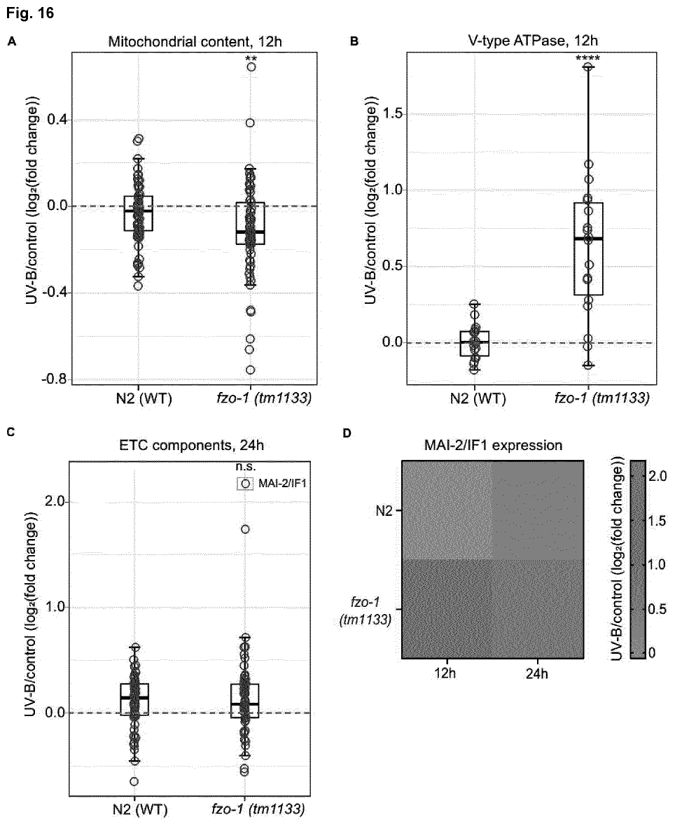

FIG. 16: UV exposure elicits changes of mitochondrial and lysosomal proteomes.

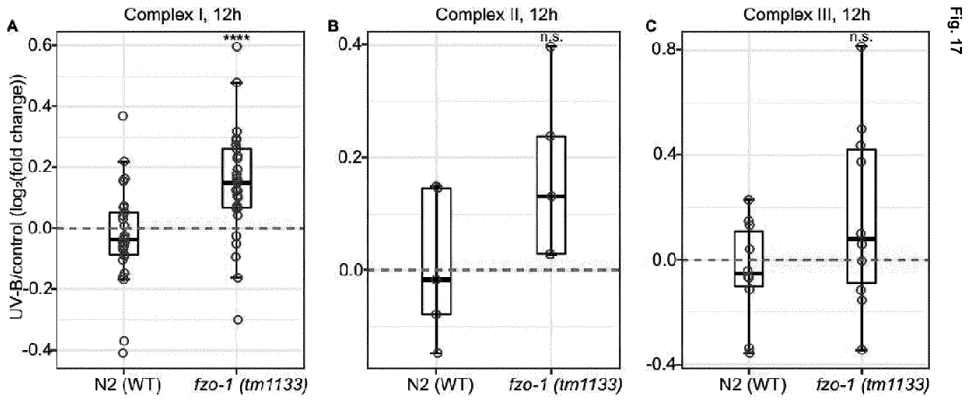

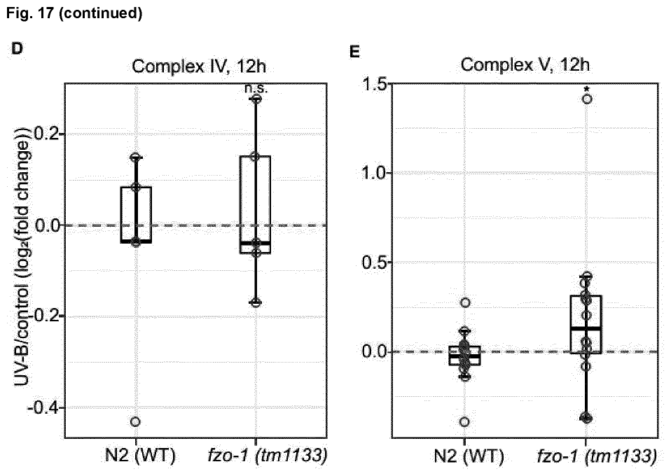

FIG. 17: UV triggers coordinated upregulation of ETC complexes I-V.

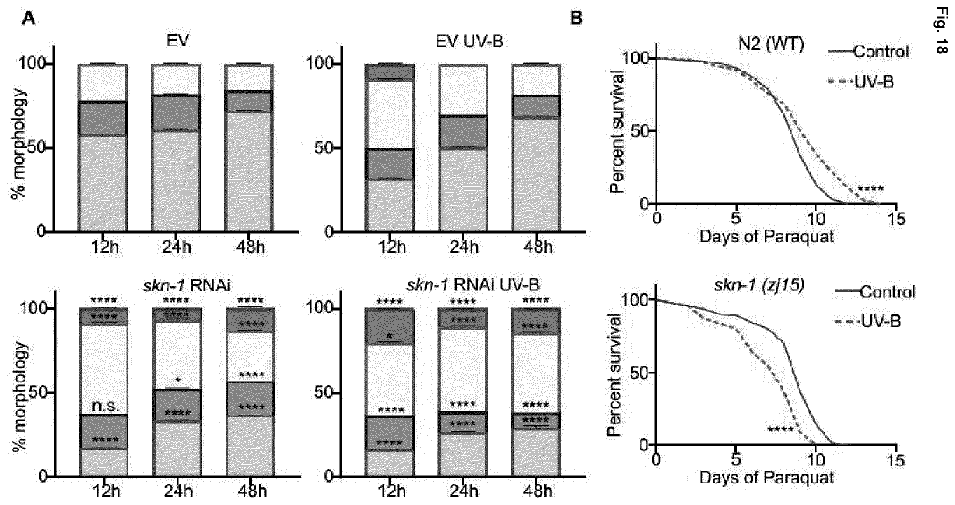

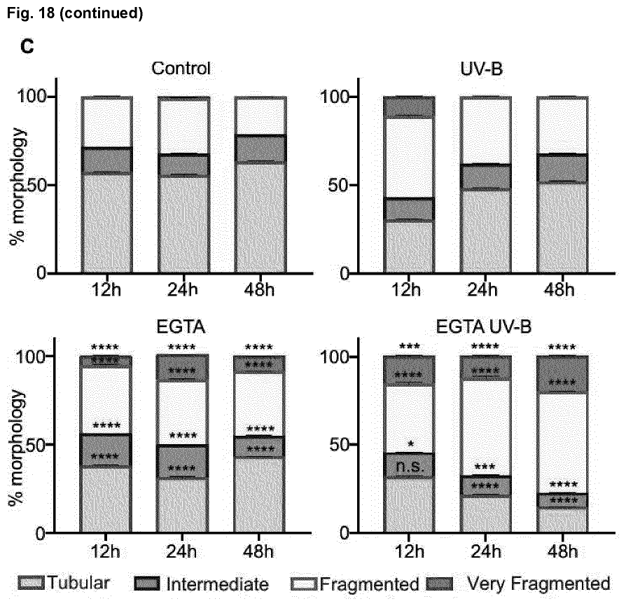

FIG. 18: UV-induced adaptive benefits require mitochondrial biogenesis and Ca2+ signaling.

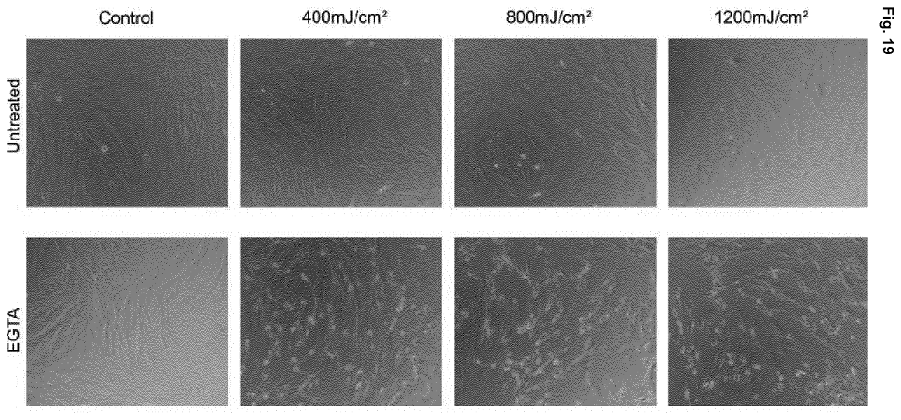

FIG. 19: Ca2+ depletion sensitizes human skin fibroblasts to UV toxicity.

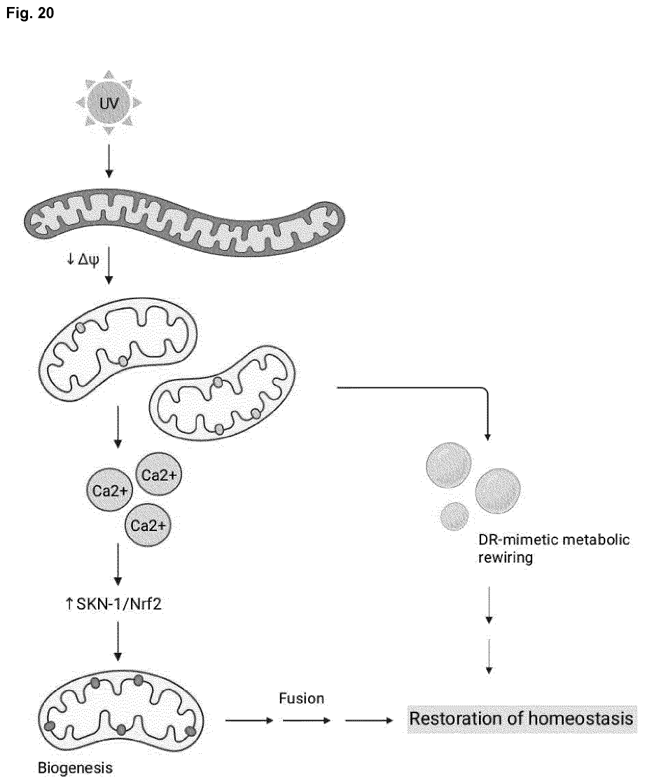

FIG. 20: A model of UV-induced metabolic rewiring response.

### DETAILED DESCRIPTION OF THE FIGURES

FIG. 1: Analysis in C. elegans. (A) Wild-type (N2 Bristol strain), drp-1 (tm1108), and fzo-1 (tm133) strains were pre-treated with UV-B (850 mj/cm2) and IR (90Gy) at L4 larval stage and 48 h later transferred to NGM plates containing 5 mM Paraquat (Sigma-856177), survival was scored daily. (B) Wild-type, drp-1 (tm1108), and fzo-1 (tm133) strains were pre-treated with UV-B and IR as in (A) and transferred to NGM plates containing 10 mM DTT (Sigma-DO632), survival was scored daily. (C) myo-3p::gfpmit transgenic animals expressing GFP tagged mitochondria in the body wall muscle of C. elegans were treated with UV-B and IR on the 1st day of adulthood. The presence of tubular, intermediate, fragmented, and very fragmented mitochondrial morphologies was scored after 12 h, 24 h, and 48 h of treatment. (D) myo-3p::gfpmit transgenic animals were grown on EV and fzo-1 RNAi bacteria from the L1 stage. The nematodes were treated with UV-B and IR on the 1st day of adulthood, and the presence of tubular, intermediate, fragmented, and very fragmented mitochondrial morphologies was scored after 12 h, 24 h and 48 h. For A-B significance was measured by Mantel-Cox test, two-tailed p values were computed. At least three independent experiments were conducted in each case, n=140 for each experimental condition. For C-D significance was measured by a two-tailed unpaired t-test (with Welch's correction), n=60, mean and S.E.M are presented. The asterisks refer to % of respective morphology in treated animals versus time point matched control. Representative results of at least three independent experiments are shown. *p<0.05; **p<0.01; ***p<0.001; ****p<0.0001; n.s., not significant.

FIG. 2: Analysis in C. elegans. (A) Representative images of the Oil Red O whole-body lipid staining are shown for wild-type control and wild-type UV-B (850 mj/cm2) treated animals. The scale bar is 200 μm. B-C Whole-body lipid content was measured by Oil Red O (ORO) staining in N2 wild-type (B) and fzo-1 (tm133) mitochondrial fusion mutant (C) strains. Strains were treated with UV-B (850 mj/cm2) and IR (90Gy) on the 1st day of adulthood and lipid content was measured after 12 h and 24 h. In Oil Red O quantification, mean gray values (ImageJ software) were used as arbitrary units (a.u.) in all cases. The asterisks show the differences between treated and control groups of respective time points. D-E Whole-body ATP levels were measured in wild-type (D) and fzo-1 (tm133) mutant (E) strains treated with UV-B (850 mj/cm2) and IR (90Gy) on adulthood day 1. ATP measurements were conducted after 12 h and 24 h post-treatment. The ATP levels are normalized to respective control for each time point. The asterisks represent the differences between treated versus control groups. For B-C n>20 for each condition and mean and S.E.M are presented. A two-tailed unpaired t-test (with Welch's correction) was used for statistical analysis. For D-E n>50 for each condition and mean and S.E.M are presented. For statistics, a two-tailed unpaired t-test (with Welch's correction) was used. For A-E representative results of at least three independent experiments are shown. *p<0.05; **p<0.01; ***p<0.001; ****p<0.0001; n.s., not significant.

FIG. 3: Analysis in C. elegans. Young adult wild type (N2 Bristol strain) and fzo-1 (tm133) mutant animals were treated with UV-B (850 mj/cm2) and protein expression was assessed by mass spectrometry after 24 h. A-C Box plots showing log 2 fold changes in UV-B exposed condition over timepoint matched untreated control for specific proteins involved in (A) glycolysis, (B) lipid storage and (C) mitochondrial lipid beta-oxidation are presented. Each dot represents an individual protein. (D) Heat map of selected proteins involved in peroxisomal beta-oxidation is shown. The color code shows log 2 (fold change) values. Four independent pools of n=800 worms were analyzed for each condition. Wilcoxon rank-sum test and two-tailed p values were used for the statistical analysis. *p<0.05; **p<0.01; ***p<0.001; ****p<0.0001; n.s., not significant.

FIG. 4: Analysis in C. elegans. (A) Young adult transgenic animals expressing GFP under the promoter of hsp-6 gene, were treated with UV-B (850 mj/cm2) and IR (90Gy). GFP fluorescence was measured by microscopy and quantified after 12 h, 24 h, and 48 h. Mip-1 RNAi was used as a positive control for UPR MT induction. Representative results of at least three independent experiments are shown. n=20 worms were analyzed for each condition. Significance was measured by a two-tailed unpaired t-test (with Welch's correction); mean and S.E.M are presented. The asterisks represent differences of treated groups over time point matched untreated controls. (B) Box plot showing fold changes of selected mitochondrial proteins upon UV-B treatment is presented. Young adult wild type (N2) and fzo-1 (tm133) mutant animals were treated with UV-B (850 mj/cm2) and levels of mitochondrial proteins were measured by mass spectrometry after 24 h. Each dot represents an individual protein. Four independent pools of n=800 worms were analyzed for each condition. Wilcoxon rank-sum test and two-tailed p values were used for statistical analysis. (C) Wild-type animals and pink-1 (tm1779) mitophagy mutants were pre-treated with UV-B (850 mj/cm2) and IR (90Gy) as in FIG. 1B and later transferred to 10 mM DTT (Sigma-DO632) containing plates, survival was scored daily. Significance was measured by Mantel-Cox test, two-tailed p values were computed. At least three independent experiments were conducted in each case, n=140 for each experimental condition. *p<0.05; **p<0.01; ***p<0.001; ****p<0.0001; n.s., not significant.

FIG. 5: Adaptive UV effects involve Δψ distortion, mitochondrial biogenesis and Ca2+ signaling. Analysis in C. elegans. Protein samples were collected as described in FIGS. 3 and 4. (A) Box plots show average Log 2 expression fold changes of selected ETC components at 12 h post exposure to 300 mJ/cm2 UV-B. Fold changes were calculated in UV-B treated versus time point matched control groups. Four independent pools of n=800 worms were collected and analyzed for each condition. Red circles represent individual proteins, and box plot parameters are as described in FIG. 3 (A-C). Wilcoxon rank-sum test and two-tailed p values were used for statistics. Asterisks compare fzo-1 (tm1133) and corresponding wild-2 type N2 samples. Purple rectangle highlights MAI-2/IF1 protein. (B) Wild-type (N2 Bristol strain) and skn-1 (zj15) mutant animals were pre-treated with UV-B (850 mJ/cm2) at L stage and after 48 h transferred to 10 mM DTT (Sigma-DO632) plates; survival was scored daily. Significance was measured by Mantel-Cox test, and two-tailed p values were computed. Each group consisted of n=140 worms. (C) Transgenic animals expressing calcium sensor GCaMP3 in body wall muscle were treated with UV-B (850 mJ/cm2) and IR (90Gy) at young (non-gravid) adult stage, and fluorescence was quantified after 6 h, 12 h, and 24 h. n>20 worms were analyzed for each condition. Significance was measured by a two-tailed unpaired t-test (with Welch's correction); mean and S.E.M values are presented. Asterisks compare treated and untreated groups at each time point. (D) PD41 human skin fibroblasts were pre-treated with 400 mJ/cm2, 800 mJ/cm2 and 1200 mJ/cm2 of UV-B, and later incubated in presence of 2 mM EGTA. Mitochondrial membrane potential was measured by JC-1 assay after 24 h. Significance was assessed by a two-tailed unpaired t-test (with Welch's correction); mean and S.E.M values are presented. Asterisks compare respective EGTA plus and minus conditions. (E) (myo-3p::gfpmit) transgenic animals expressing GFP-tagged mitochondria in the body wall muscle were pretreated with indicated amounts of Rapamycin (R-5000, LC laboratories) for 24 hours before UV-B (850 mJ/cm2) treatment on adulthood day 1 (AD1). The % of tubular, intermediate, fragmented, and very fragmented mitochondria were scored after 12 h. Significance was measured by a two-tailed unpaired t-test (with Welch's correction). n=60 in each condition, mean and S.E.M values are presented. Asterisks compare respective UV-B treated and untreated groups. *p<0.05; **p<0.01; ***p<0.001; ****p<0.0001; n.s., not significant. In B-D representative results of at least three independent experiments are shown.

FIG. 6: Analysis in C. elegans. (A) Young (adulthood day 1, AD1) and old (adulthood day 10, AD10) transgenic animals expressing GFP tagged mitochondria in the body wall muscle (myo-3p::gfpmit) were treated with UV-B (850 mj/cm2). The presence of tubular, intermediate, fragmented, and very fragmented mitochondrial morphologies was scored after 12 h, 24 h, and 48 h. Significance was measured by a two-tailed unpaired t-test (with Welch's correction), n=60, mean and S.E.M are presented. The asterisks refer to % of respective morphology in treated animals versus time point matched control. Young (AD1) (B) and old (AD10) (D) wild-type animals were treated with UV-B (850 mj/cm2) and transferred to 10 mM DTT plates after 48 h, survival was scored daily. (C) Old (AD10) wild-type worms were treated with UV-B (850 mj/cm2) and scored after 24 h and 48 h for survival with no additional stressors applied. For B-D significance was measured by Mantel-Cox test, two-tailed p values were computed. At least two independent experiments were conducted in each case, n=140 for each experimental condition. *p<0.05; **p<0.01; ***p<0.001; ****p<0.0001; n.s., not significant. (E) Summary model is shown: UV-B treatment of young animals induces transient mitochondrial fragmentation leading to a systemic dietary restriction-like metabolic rewiring and conferring increased stress resilience of the animals. The metabolic rewiring response features a prominent lipid turnover component (TAGs).

FIG. 7: Analysis in C. elegans. (A) The graphical summary of stress adaptation tests performed in wild-type and mutant C. elegans strains is presented. Nematodes were treated with ionizing radiation (IR) and UV-B light and left at 20°° C. for 48 h to ensure DNA damage repair. Subsequently, the animals were transferred to Paraquat (5 mM) plates to induce oxidative stress or to DTT (10 mM) plates to induce unfolded protein stress in the endoplasmic reticulum. After the transfer, survival was scored daily. (B) To identify optimal UV-B dose, which induces adaptive stress responses, the worms were treated at the L4 stage with different doses of UV-B and IR (90Gy). After 48 h worms were treated with subsequent heat stress. UV-B 850 mj/cm2 and IR 90Gy (described previously) were determined as optimal doses for further stress assays. Significance was measured by Mantel-Cox test, two-tailed p values were computed. Three independent experiments were conducted, n≥100 for each experimental condition. *p<0.05; **p<0.01; ***p<0.001; ****p<0.0001; n.s., not significant. (C) Representative pictures of tubular, intermediate, fragmented and very fragmented mitochondria are shown in transgenic animals expressing GFP-labelled mitochondria.

FIG. 8: Analysis in C. elegans. (A) Representative images of Oil Red O (ORO) staining in wild type and fzo-1 (tm133) mutant animals are presented. Worms were treated with UV-B (850 mj/cm2) and IR (90Gy) and microscopy of stainings was performed after 12 h and 24 h. The scale bar is 200 μm. (B) Whole-body lipid content was measured by Oil Red O (ORO) staining in N2 wild-type worms. The animals were treated with high doses of UV-B (1250 mj/cm2 and 1500 mj/cm2) and lipid content was assessed after 12 h and 24 h. In Oil Red O quantification, mean gray values (ImageJ software) were used as arbitrary units (a.u.) in all cases. The asterisks show the differences between treated and control groups of respective time points. n>20 for each condition and mean and S.E.M are presented. A two-tailed unpaired t-test (with Welch's correction) was used for the statistical analysis. Representative results of at least three independent experiments are shown. *p<0.05; **p<0.01; ***p<0.001; ****p<0.0001; n.s., not significant. (C) Representative pictures of Oil Red O (ORO) staining of wild-type worms treated with high doses of UV-B (1250 mj/cm2 and 1500 mj/cm2) are shown. Lipid staining was measured and microscopy was performed after 12 h and 24 h. The scale bar is 200 μm.

FIG. 9: Analysis in C. elegans. Young adult wild type and fzo-1 (tm133) mutant animals were treated with UV-B (850 mj/cm2) and levels of specific proteins were measured by mass spectrometry after 24 h. A-B Box plots showing fold changes for specific proteins involved in (A) immune response and (B) proteostasis are presented. Each dot corresponds to an individual protein. Log2 fold changes were calculated in UV-B treated over timepoint matched control animals. Four independent pools of n=800 worms were analyzed for each condition. Wilcoxon rank-sum test and two-tailed p values were used for statistical analysis. *p<0.05; **p<0.01; ***p<0.001; ****p<0.0001; n.s., not significant.

FIG. 10: Analysis in C. elegans. Representative images of the transgenic strain expressing GFP under control of the hsp-6 gene promote treated with UV-B (850 mj/cm2) and IR (90Gy) at the young adult stage are shown. Mip-1 RNAi was used as a positive control that induces UPR MT. The microscopy pictures were taken at 12 h, 24 h and 48 h post-treatment with Zeiss AxioZoom.V16. Exposure time for the GFP channel was 20 ms in all cases. Magnification was set to 100×, imaging device—Axiocam 503.

FIG. 11: Analysis in C. elegans. Representative images of the transgenic strain expressing GFP under control of the hsp-4 gene promote treated with UV-B (850 mj/cm2) and IR (90Gy) at the young adult stage are shown. 1,4-Dithiothreitol (DTT) was used as a positive control that induces UPR ER via inhibiting the formation of disulfide bonds. The microscopy pictures were taken 6 h, 12 h, and 24 h post-treatment with Zeiss AxioZoom.V16. Exposure time for the GFP channel was 30 ms in all cases. Magnification was set to 80×, imaging device—Axiocam 503.

FIG. 12: (A) Overview of proposed mechanism: At a young age treatment with UV-B leads to a dietary restriction-like metabolic rewiring via transient mitochondrial fragmentation. Due to these metabolic changes, young organisms become more stress adaptive following moderate UV-B exposure. Mitochondrial fusion plays a central role in the metabolic benefits of UV-B treatment. Conversely, aging-associated and congenital defects in mitochondrial fusion abrogate the positive effects of UV-B-induced metabolic rewiring and sensitize old animals to UV-B toxicity. (B) This image illustrates one of the problems the present invention had to solve, namely to determine a safe and efficient dose of UV-B that is high enough to elicit metabolic effects but not too high to avoid permanent cell damage.

FIG. 13: UV-induced mitochondrial fragmentation is exacerbated by mitofusin gene knock down. Analysis in C. elegans. (A) L4 stage N2 wild-type worms were treated with different doses of UV-B (500 mJ/cm2, 850 mJ/cm2 and 1500 mJ/cm2) and IR (90Gy). After 48 h at 20° C. worms were treated with heat stress (35° C.) and survival was scored at 2 h, 4 h, 6 h, 7 h, 8 h post-exposure. UV-B 850 mJ/cm2 and IR 90Gy were determined as optimal doses for further stress assays. Significance was measured by the Mantel-Cox test, and two-tailed p values were computed. At least three independent experiments were conducted in each case, n>100 for each experimental condition. (B) Representative images of tubular, intermediate, fragmented, and very fragmented mitochondria in transgenic animals expressing GFP labelled mitochondria in the body wall muscle (myo-3p::gfpmit). The scale bar is 20 μm. (C) (myo-3p::gfpmit) transgenic animals were grown on EV and fzo-1 RNAi bacteria from the L1 stage. The nematodes were treated with UV-B (850 mJ/cm2) on adulthood day 1 (AD1) and % of tubular, intermediate, fragmented, and very fragmented mitochondria were scored 12 h, 24 h and 48 h post-exposure. Significance was measured by a two-tailed unpaired t-test (with Welch's correction), n=60 in each condition, mean and S.E.M values are presented. The asterisks compare respective morphologies of fzo-1 RNAi nematodes with time point-and treatment-matched EV control. Representative results of at least three independent experiments are shown. *p<0.05; **p<0.01; ***p<0.001; ****p<0.0001; n.s., not significant.

FIG. 14: Mitochondrial and nuclear effects of UV-B are independent. Analysis in C. elegans. UV treatment and proteomics sample collection was carried out as described in FIG. 3. Box plots showing average Log 2 expression fold changes of selected C-type lectins (A) and heat shock proteins (B) are presented. Fold changes were calculated between UV-B treated and respective time-point matched control groups. Red circles represent individual proteins and box plot parameters are as described in FIG. 3 (A-C). Four independent pools of n=800 worms were analyzed for each condition. Wilcoxon rank-sum test and two-tailed p values were used for statistical analysis. Asterisks compare respective fzo-1 (tm1133) and wild-type N2 samples. (C) Transgenic animals expressing GFP-tagged mitochondria in the body wall muscle (myo-3p::gfpmit) were grown on EV and rad-23 RNAi bacteria from the L1 stage and treated with UV-B (850 mJ/cm2) on adulthood day 1; the % of tubular, intermediate, fragmented, and very fragmented mitochondria were scored at 12 h, 24 h and 48 h post-exposure. Significance was measured by a two-tailed unpaired t-test (with Welch's correction), n=60 in each condition, mean and S.E.M values are presented. The asterisks compare respective morphologies of rad-23 RNAi nematodes with time point-and treatment-matched EV control. Representative results of at least three independent experiments are shown. *p<0.05; **p<0.01; ***p<0.001; ****p<0.0001; n.s., not significant.

FIG. 15: Moderate UV and IR treatments do not activate mitochondrial UPR. Analysis in C. elegans. Representative images of the transgenic animals expressing GFP under control of the hsp-6 mitochondrial chaperone gene promoter and treated with UV-B (850 mJ/cm2) and IR (90Gy) at young (non-gravid) adult stage. mip-1 RNAi was used as a positive control that induces UPRMT. The microscopy pictures were taken at 12 h, 24 h and 48 h post treatment. The scale bar is 200 μm.

FIG. 16: UV exposure elicits changes of mitochondrial and lysosomal proteomes. Analysis in C. elegans. UV treatment and proteomics sample collection was carried out as described in FIG. 3. Box plots showing average Log 2 expression fold changes of (A) selected mitochondrial ribosome proteins, (B) V-type ATPase components and (C) ETC components at indicated times following UV exposure are presented. In all cases, fold changes were calculated between UV-treated and respective time point matched control samples. In C, purple rectangle highlights MAI-2/IF1 protein. Red circles represent individual proteins and box plot parameters are as described in FIG. 3 (A-C). Four independent pools of n=800 worms were analyzed for each condition. Wilcoxon rank-sum test and two-tailed p values were used for statistical analysis. Asterisks compare respective fzo-1 (tm1133) and wild-type N2 samples. *p<0.05; **p<0.01; ***p<0.001; ****p<0.0001; n.s., not significant. (D) Heatmap of the MAI-2/IF1 protein expression in N2 and fzo-1 (tm1133) strains at 12 h and 24 h post UV exposure is shown. The color bar depicts log 2 expression fold change values.

FIG. 17: UV triggers coordinated upregulation of ETC complexes I-V. Analysis in C. elegans. UV treatment and proteomics sample collection was carried out as described in FIG. 3. Box plots showing average Log 2 expression fold changes of selected components of (A) Complex I (B) Complex II (C) Complex III (D) Complex IV and (E) Complex V at 12 h post exposure to 300 mJ/cm2 UV-B. In all cases, fold changes were calculated between UV-treated and respective time point matched controls samples. Red circles represent individual proteins and box plot parameters are as described in FIG. 3 (A-C). Four independent pools of n=800 worms were analyzed for each condition. Wilcoxon rank-sum test and two-tailed p values were used for statistical analysis. Asterisks compare respective fzo-1 (tm1133) and wild-type N2 samples. *p<0.05; **p<0.01; ***p<0.001; ****p<0.0001; n.s., not significant.

FIG. 18: UV-induced adaptive benefits require mitochondrial biogenesis and Ca2+ signaling. Analysis in C. elegans. (A) Transgenic animals expressing GFP-tagged mitochondria in the body wall muscle (myo-3p::gfpmit) were grown on EV and skn-1 RNAi from L1 stage and exposed to 850 mJ/cm2 UVB on adulthood day 1 (AD1). Mitochondrial morphology was scored at 12 h, 24 h and 48 h post UV-B treatment. Two-tailed unpaired t-test (with Welch's correction) was used for the statistics. n=60 in each condition, mean and S.E.M values are presented. The asterisks compare respective morphologies of skn-1 RNAi nematodes with time point-and treatment-matched EV control. (B) Wild-type (N2 Bristol strain) and skn-1 (zj15) mutant animals were pre-treated with 850 mJ/cm2 UV-B at L4 stage and after 48 h transferred to 5 mM Paraquat (Sigma-856177) plates; survival was scored daily. Significance was measured by the Mantel-Cox test, and two-tailed p values were computed. Each group consisted of n=140 worms. (C) (myo-3p::gfpmit) transgenic animals were treated with UV-B (850 mJ/cm2) on AD1 and immediately picked onto plates containing 50 mM EGTA. The % of tubular, intermediate, fragmented, and very fragmented mitochondria were scored after 12 h, 24 h and 48 h. Significance was measured by a two-tailed unpaired t-test (with Welch's correction). n=60 in each condition, mean and S.E.M values are presented. The asterisks compare respective morphologies of EGTA plus nematodes with time point-and treatment-matched EGTA minus control. In A-C representative results of at least three independent experiments are shown.*p<0.05; **p<0.01; ***p<0.001; ****p<0.0001; n.s., not significant.

FIG. 19: Ca2+ depletion sensitizes human skin fibroblasts to UV toxicity. Analysis in C. elegans. Representative microscopy images of human skin fibroblasts 24 h post-treatment with 400 mJ/cm2, 800 mJ/cm2 and 1200 mJ/cm2 of UV-B and incubation in presence or absence of 2 mM EGTA.

FIG. 20: A model of UV-induced metabolic rewiring response. UV-B light triggers mitochondrial network fragmentation and Ca2+ release via disruption of OXPHOS and distortion of mitochondrial Δψ. Ca2+ activates mitochondrial biogenesis via SKN-1/Nrf2, and newly generated ETC-rich mitochondria are integrated into the network by fusion to restore healthy homeostasis without lasting mitochondrial damage. This UV recovery process is paralleled by transient DR-mimetic metabolic rewiring, which warrants therapeutic exploration. Created with BioRender.

### EXAMPLES

The invention is further described by the following examples. These are not intended to limit the scope of the invention but represent preferred embodiments of aspects of the invention provided for greater illustration of the invention described herein.

In the present example the inventors tested the impact of mitochondrial adaptations on the cellular adaptive stress responses to UV-B and IR. The present experiment inquires if inhibition of either of these mitochondrial responses would have a negative impact on UV-B and IR induced stress tolerance of the model organism C. elegans, used as a representative for a human subject. Initially, L4 stage animals were exposed to 90Gy IR and different doses of UV-B and were then treated 48 h later with heat stress, as described previously, to identify the optimal UV-B treatment conditions for the induction of adaptive stress tolerance (FIG. 7A and 7B, FIG. 13A). These results demonstrate that an optimal sub-erythemal dose of UV-B is required to achieve a positive effect, without inducing adverse effects related to UV-toxicity.

The experiments next confirmed the ability of the chosen UV-B and IR doses to provide protection from stress by replacing heat stress with common physiological stressors such as oxidative stress (induced by paraquat) and ER protein folding stress (induced by DTT) (FIG. 1A and 1B). Subsequently it was assessed if inhibition of mitochondrial fission by loss of the DNM1L orthologue drp-1 and fusion by loss of the mitofusin orthologue fzo-1 would impact UV-B-and IR-induced stress tolerance. The experiment revealed that drp-1 was dispensable for these responses (FIG. 1A and B), while lack of mitochondrial fusion strikingly sensitized the animals to stress following treatment with UV-B but not IR (FIG. 1A and B), suggesting that mito-fusion (mitochondrial fusion) is essential for the maintenance of systemic homeostasis following UV-B exposure. Consistently, UV-B but not IR induced persistent mitochondrial fragmentation in transgenic animals expressing GFP-labelled mitochondria (FIG. 1C and 7C), which was exacerbated by RNAi-mediated knock down of fzo-1 (FIG. 1D and FIG. 13C). Accordingly, it was shown that UV-B treatment induces rapid mitochondrial fragmentation in vivo, which needs to be mitigated by the fusion machinery to maintain healthy homeostasis.

In previous work (Espada et al, 2020) the inventors found, that transient impairment of mitochondrial function results in a dietary restriction-like metabolic rewiring, particularly it leads to a DR-like lipid turnover response, which can be measured by the whole-body Oil Red O (ORO) staining. In the present example it was inquired next if UV-B treatment caused such a DR-like response. The results show that in wild type animals UV-B indeed induces a transient decline of whole-body lipid content, which recovers to baseline levels within 24 h post exposure (FIG. 2A and 2B). This experiment demonstrates the ability of moderate UV-B exposure to trigger a reduction of whole-body lipid content. The ORO assay is a way to measure whole body lipid content via microscopy. The figures also demonstrate the metabolic recovery/plasticity phenotype seen by the return of ORO staining to control levels after 24 h post UV-B treatment.

Importantly, lipid levels did decline but did not recover in UV-B exposed fzo-1 mutant animals (FIG. 2C and 8A), in line with the key roles of mitochondrial fragmentation and fusion in the DR-like effect of UV-B uncovered by this study. Additionally, at higher UV-B doses the recovery of lipid levels following UV exposure was abrogated also in wild type animals (FIG. 8B and 8C), clearly showing that a particular window of UV doses is effective in eliciting the DR-like effects without permanent distortion of metabolism. In line with the ability of UV-B to cause the DR-like impact on the systemic metabolism, the results evidence the ability of UV-B to elicit a transient drop in whole-body ATP levels in N2 animals (FIG. 2D), which was again more persistent in mitofusin mutants (FIG. 2E). Accordingly, it is important to incorporate the recovery phase (break between treatments) into the human cosmetic treatment regimen because lack of recovery leads to UV toxicity, as seen in UV sensitive fzo-1 mutants (combining lack of ORO recovery seen in FIGS. 2C and 8A with the UV-induced lethality of the fzo-1 mutant background seen in FIG. 1A-B).

The inventors next performed proteomics analysis in WT and mitofusin mutant animals to test for molecular activities associated with dietary restriction and metabolic rewiring. Consistent with the DR-like effects of UV-B, the results showed indeed the upregulation of glycolysis (FIG. 3A), downregulation of lipid droplet components (FIG. 3B) and upregulation of beta oxidation enzymes both mitochondrial (FIG. 3C) and peroxisomal (FIG. 3D) in wild type animals exposed to UV-B. Comparable responses can be assessed by measuring glucose levels, LDL, HDL and triglyceride levels in human blood samples. For example, a reduction of LDL and an increase of HDL levels is expected upon UV-B treatment in this case. Strikingly, all of these rewiring activities were abrogated and even reversed in UV-B-treated mitofusin mutant animals (FIG. 3A-D), in line with the key role of mitochondrial fusion in the recovery of metabolic homeostasis following UV-B exposure.

Previous work of the inventors (Ermolaeva et al, 2013) revealed that nuclear DNA damage inflicted by UV-B and IR was linked to systemic stress tolerance by elevated innate immune signaling and enhanced proteostasis. Here the induction of both effects by UV-B could be detected through proteomics analysis, and they were not abrogated in mitofusin mutant animals (FIG. 9A and B, FIG. 14A and B). Moreover, a previous study found that persistent occurrence of helix distorting nuclear DNA lesions (the damage kind induced by UV-B) leads to mitochondrial hyper-fusion and not fragmentation (Lopes A et al, Nucleic Acids Res 2020). Taken together, the present results suggest that nuclear and mitochondrial effects of UV-B are likely independent.

The inventors next asked if UV-B disturbs mitochondrial homeostasis by inflicting damage of mitochondrial DNA and/or mitochondrial proteins. Both these damages converge in triggering mitochondrial unfolded protein response, which can be visualized in vivo by hsp-6p::GFP reporter. The inventors found however that neither UV-B nor IR triggered detectable UPR MT induction at their beneficial doses used in this study (FIG. 4A, 10 and 16). Subsequently, a reduction of mitochondrial content was not observed in UV-B exposed wild type animals by proteomics (FIG. 4B), and mutants lacking key mitophagy mediator PINK-1 did not fail in developing UV-B-and IR-induced stress tolerance (FIG. 4C).

Collectively, these results demonstrate that moderate UV-B treatment does not cause lasting damage to mitochondrial DNA and proteome in wild type animals.

Further, it does not trigger mitochondrial elimination by mitophagy in these animals. Conversely, in mitofusin mutants UV-B treatment led to a significant decline of mitochondrial content (FIG. 4B), demonstrating that mitochondrial fusion is protective against lasting negative impact of UV-B exposure on mitochondria.

Because the present tests ruled out direct DNA and protein damage as instigators of mitochondrial fragmentation by UV-B, the inventors next investigated what other aspects of mitochondrial homeostasis could be direct UV-B targets. Previous data hinted towards the ability of UV to directly impair the activity of mitochondrial ATP synthase by causing conformational changes in its active center (Chavez E and Cuellar A, Arch Biochem Biophys. 1984; Beyer RE, Biochim Biophys Acta. 1962), and pharmacological ATP synthase inhibition by drugs like oligomycin was previously found to trigger the disruption of the mitochondrial network similar to the effect of UV-B observed in FIG. 1C (Kim JM, Anim Cells Syst. 2018). In addition to changes of mitochondrial morphology, the alterations of electron transport, expected to occur upon ATPase impairment, are known to trigger shifts in mitochondrial calcium uptake, causing depletion of calcium from the ER (Mbaya E, Cell Death Differ. 2010) and subsequent ER protein folding stress.

The inventors next tested if UV-B exposure triggers the activation of UPR ER by using transgenic animals that express hsp4p::gfp reporter and found this to be the case (FIG. 11A).

Collectively, the results reveal that moderate doses of UV-B are able to inflict DR-like metabolic remodeling by inducing mitochondrial fragmentation, and mito-fusion is required to ensure the transient nature of this response and avoid lasting mitochondrial damage and reduced survival of UV-B-exposed animals. Concurrently, previous work demonstrated that the efficacy of mito-fusion significantly declines with age with impact on the systemic competence of metabolic adaptation (Espada et al, 2020 and others). The inventors thus asked if aging affected the capacity of animals to recover from mitochondrial stress induced by UV-B. While mitochondrial fragmentation was transient in UV-B-exposed young animals, it was more pronounced and more persistent in the old ones (FIG. 6A). Consistently, while young animals benefited from UV-B exposure by developing adaptive stress tolerance (FIG. 6B), the survival of old UV-B-treated animals was severely reduced both without stress (FIG. 6D) and upon additional ER stress exposure (FIG. 6C). The present example thus demonstrates that aging-associated decline of mito-fusion capacity abrogates metabolic benefits of UV-B in old animals and, on the contrary, sensitizes them to metabolic damage driven by persistent mitochondrial fragmentation in a manner, which resembles the detrimental response of mitofusin mutants to UV-B. Accordingly, it can be assumed that such a UV-B intervention is not entirely safe and becomes toxic in late life, wherein AD10 (adulthood day 10) age in nematodes is comparable to ≥65 years in humans.

In summary, UV light is a common environmental factor, which affects humans regularly. UV exposure has been proposed to elicit benefits for systemic homeostasis and metabolism, but their mechanism is not well understood. The present examples show, by using C. elegans, that UV elicits metabolic benefits by acting as a dietary restriction (DR) mimetic at the systemic and molecular levels. Mechanistically, moderate UV exposure causes a rapid disruption of the mitochondrial network. This effect is not accompanied by lasting damage of mtDNA and proteins and requires fusion machinery to orchestrate the metabolic recovery process, which closely resembles DR. Importantly, the present example demonstrates that aging-associated defects in mito-fusion not only abrogate systemic UV benefits in late life but also sensitize old organisms to direct UV toxicity. These findings have implications for the use of UV as an accessible metabolic intervention and for the safety of recreational UV exposure in late life.

Collectively the present example finds that moderate UV-B exposure induces mitochondrial fragmentation/fission through direct interference with mitochondrial bioenergetics capacity, eliciting a transient metabolic remodeling response, which resamples dietary restriction both at the systemic and molecular levels (FIG. 6E). The mitochondrial fusion machinery is required for the transient nature of this response and for ensuring its lasting benefits in terms of enhanced homeostasis and stress tolerance. The inventors herein found that defects in mitochondrial fusion, which occur because of mutations or due to aging (e.g. at a C. elegans age, which is comparable to a human age of over 65), abrogate the metabolic benefits of UV-B treatment and sensitize the organisms to UV-B toxicity (FIG. 12A). Hence, the present example thus demonstrates that UV-B acts as an accessible and effective dietary restriction mimetic, and aging (>65 y) is identified herein as a risk factor of metabolic UV-B toxicity, keeping in mind that UV light is one of the most common environmental factors, which affects humans on a daily basis.

Homeostatic recovery upon UV exposure requires mitochondrial biogenesis and Ca2+ signaling. By performing deeper analysis of the UVB-altered mitochondrial proteome, the inventors found that the expression of electron transport chain (ETC) components (Espada et al., 2020) was prominently elevated in mitofusin mutants at 12 h post UVB exposure (FIG. 5A) despite the overall decline of mitochondrial content seen in these animals (FIG. 4B). This phenotype held true for each individual ETC complex with highest significance seen for complexes I and V, possibly due to bigger number of components detected for these complexes (FIG. 17A-E). This observation suggested that the distortion of mitochondria by UVB might trigger the biogenesis of ETC-enriched organelles, which are integrated into the network via fusion to restore healthy metabolism and preserve mitochondrial integrity. In C. elegans, mitochondrial biogenesis is regulated by the conserved transcription factor SKN-1/Nrf2 (Palikaras et al., 2015a), and inactivation of this gene by mutation or RNAi indeed prevented the recovery of the mitochondrial network following UVB exposure (FIG. 18A) and sensitized the worms to UVB toxicity (FIG. 5B and 18B). Because SKN-1 was shown to respond to elevation of cytosolic Ca2+ levels triggered by mitochondrial dysfunction (Palikaras et al., 2015a, b), the inventors next exposed the animals to Ca2+ chelator EGTA (Palikaras et al., 2015a) and indeed found that Ca2+ removal prevented the recovery of mitochondrial integrity in UVB-exposed animals similar to phenotypes seen upon RNAi-mediated inactivation of skn-1 and fzo-1 (FIG. 18C). Consistently, transient Ca2+ release could be detected in vivo at 12 h post UVB exposure by using transgenic animals expressing Ca2+ sensor GCaMP3 in body wall muscle (Schwarz et al., 2012) (FIG. 5C). Moreover, UVB-treatment of human primary skin fibroblasts (a relevant cell type with regard to UV effects in humans (Rognoni et al., 2021)) did not lead to a measurable disruption of mitochondrial homeostasis unless it was combined with EGTA exposure, in which case UV promoted loss of mitochondrial membrane potential (MMP) and cell death (FIG. 5D and 19).

Because UVB treatment led to the coordinated upregulation of ETC components (FIG. 17A-E), the inventors hypothesized that this could be a response to direct distortion of oxidative phosphorylation (OXPHOS) by UVB. Interestingly, early studies in isolated mitochondria indeed demonstrated the ability of UV to impair OXPHOS (Beyer, 1959; Dallam and Hamilton, 1964), thus interfering with mitochondrial proton gradients, which in turn can trigger mito-fragmentation (Miyazono et al., 2018) and Ca2+ release (Zhao et al., 2013). The UV-induced distortion of mitochondrial function could be alleviated by direct provision of ATP to the organelles (Beyer, 1961), consistent with the capacity of complex V to act as a proton pump for Δψ stabilization by switching from ATP synthesis to ATP hydrolysis (Chinopoulos et al., 2010; Ichikawa et al., 1990). Strikingly, the highest upregulated respiratome component in UVB-exposed mitofusin mutants was MAI-2 (FIG. 5A), the C. elegans ortholog of the inhibitory factor 1 (IF1) (Fernandez-Cardenas et al., 2017), and MAI-2 upregulation upon UV was seen only in the mutants and not in WT animals (FIG. 17D). The role of IF1 is to inhibit the ATP hydrolysis function of complex V preventing mitochondria from causing whole cell ATP exhaustion under conditions of persistent OXPHOS failure (Campanella et al., 2008). Consistently, stabilization of the in vivo ATP content by rapamycin exposure (Espada et al., 2020) alleviated UV-induced mitochondrial fragmentation, in line with OXPHOS distortion being the primary trigger of this phenotype (FIG. 5E). The example thus reveals the surprising and previously unknown molecular mechanism, which mediates metabolic remodelling and subsequently restores healthy homeostasis following moderate UV-B irradiation in a safe dose range.

Collectively, the data obtained by the inventors supports the model that in WT animals and cells the distortion of mitochondrial bioenergetics by moderate UVB exposure triggers mitochondrial fragmentation and elicits a Ca2+ signal that initiates biogenesis of ETC-enriched mitochondria followed by their integration into the network via fusion. This recovery process prevents lasting mitochondrial damage upon UV exposure, while the initial decline of mitochondrial output triggered by UV elicits DR-like metabolic rewiring and contributes to systemic stress tolerance (FIG. 20).

1. A method for reducing body weight of a subject comprising exposing a skin area of the subject to a sub-erythemal dose of UV-B radiation.

2. The method according to claim 1, wherein the UV-B radiation is a narrow-band UV-B radiation.

3. The method according to claim 1, wherein the subject is a human.

4. The method according to claim 1, wherein the exposing is repeated on different days, with breaks of 1-7 days, until a weight loss has occurred.

5. The method according to claim 1, wherein after the exposure, a level of one or more markers selected from the group consisting of serum calcium, cholesterol, HDL cholesterol, LDL cholesterol, and triglyceride levels, serum glucose, insulin, adiponectin, leptin, blood NLR and serum interleukin 6, CRP, and IL-1β is determined in the sample obtained from said subject.

6. The method according to claim 1, wherein the exposing is repeated on 1-7 days per week.

7. The method according to claim 1, wherein the exposed skin area corresponds to at least 5% of the body surface of the subject.

8. The method according to claim 1, wherein an initial sub-erythemal dose is determined based on a skin type of the subject, wherein the initial dose for a subject with:
Fitzpatrick skin type I is 0.2 J/cm²,
Fitzpatrick skin type II is 0.3 J/cm²,
Fitzpatrick skin type III is 0.5 J/cm², or
Fitzpatrick skin type IV to VI is 0.6 J/cm².

9. The method according to claim 1, wherein the subject is 65 years old or younger.

10. The method according to claim 1, wherein the narrow-band UV-B radiation induces transient and/or reversible metabolic remodeling involving one or more of mitochondrial fragmentation/fission, peroxisomal and mitochondrial lipid beta-oxidation, lipid droplet turnover and glycolysis.

11. The method according to claim 1, wherein the method is a cosmetic method for improving the physical appearance of the subject.

12. The method according to claim 1, wherein the method is a method for treating obesity and/or metabolic syndrome in the subject.

13. The method according to claim 1, wherein a N-narrow-band UV-B lamp is employed for the treatment of obesity and/or metabolic syndrome in a subject, wherein the lamp emits UV-B radiation in the range of 305-315 nm and is configured for whole body irradiation of a human subject.

14. (canceled)

15. (canceled)

16. The method according to claim 2, wherein the narrow-band UV-B radiation is in a range of 305-315 nm.

17. The method according to claim 7, wherein the exposed skin area corresponds to at least 20% of the body surface of the subject.

18. The method according to claim 7, wherein the exposed skin area corresponds to at least 80% of the body surface of the subject.

19. The method according to claim 8, wherein for a repeated exposure, the initial dose is increased depending on skin appearance 12-24 hours after the initial exposure.

20. The method according to claim 9, wherein the subject is 50 years or younger.

21. The method according to claim 11, wherein the subject has a body mass index of no more than 25.
